(19) United States
(12) Patent Application Publication
(10) Pub. No.: US 2024/0122498 A1
(43) Pub. Date: Apr. 18, 2024

(54) DEVICE, SYSTEM AND METHOD FOR CONTINUOUS ESTIMATION OF ORGAN AND TISSUE SIZE AND CONTENT WITHIN AN INTACT ORGANISM

(71) Applicant: Yale University, New Haven, CT (US)

(72) Inventor: Pramod Bonde, New Haven, CT (US)

(21) Appl. No.: 18/486,265

(22) Filed: Oct. 13, 2023

Related U.S. Application Data
(60) Provisional application No. 63/379,319, filed on Oct. 13, 2022.

(51) Int. Cl. A61B 5/107

(52) U.S. Cl. CPC A61B 5/1073; A61B 5/1076

(57) ABSTRACT

An internal estimation system is described. The system has a flexible tube having a first resonator, the flexible tube configured to advance into an anatomical lumen of a subject. A second resonator is configured to attach to a skin surface of the subject. The first and second resonator are resonantly coupled. A controller is connected to the first resonator and the second resonator. The controller is configured to estimate a distance between the first and second resonator based on an electrical signal parameter measured by the first resonator. A method for internal estimation and a multi-dimensional estimation system and method is also disclosed.

### CROSS-REFERENCE TO RELATED APPLICATIONS

This application claims priority to U.S. provisional application No. 63/379,319, filed on Oct. 13, 2022 incorporated herein by reference in its entirety.

### BACKGROUND OF THE INVENTION

VAP (ventilator associated pneumonia) develops due to reflux of contents from the stomach and intestine into the lung, thus causing chemical injury and setting up ideal conditions for organisms to grow and cause lung damage via pneumonia. Ideally a continuous measurement of the stomach size or intestinal size can help prevent dangerous collection of residue which if not addressed in time can spill over the lungs and cause pneumonia called VAP.

Estimation and measurement of organ size, content, volume and their relation to each other over time are currently measured by X ray (with and without contrast), ultrasound, MRI, nuclear imaging, repeated suctioning of the contents, and clinical examination. These methods only give information about a particular point in time. A continuous measurement is not feasible or practical considering the size of these technologies and need for a dedicated resource.

Malnutrition leads to an increase in medical expenses, increased length of hospital stays, and poor patient prognosis. Up to 40% of in-hospital patients are affected by “disease-related malnutrition,” which is a specific type of malnutrition caused by concomitant disease (Cederholm 2017; Nutrition Day). To achieve target energy for hospitalized patients who are at risk of malnutrition, it is important to take pre-emptive measures. However, in some people with acute illness, oral intake of food may not provide the necessary nutritional value due to various reasons (Gomes 2017; Weimann 2017) which may include appetite loss due to acute illness, nausea, vomiting, early satiety, and difficulty in swallowing. This issue is especially relevant in critically ill patients since oral intake is severely affected for reasons including the need for mechanical ventilation, gastrointestinal surgery, or unconsciousness. Patients under such conditions have higher mortality rates (Esteban 2013; Rubenfeld 2005), and it is imperative for medical care providers to explore the best interventions to maintain proper nutritional status, especially since nutritional management has recently been emphasized as an important determinant of patient survival (Reintam 2017). Critically ill patients also show highly variable metabolic and immune responses to injury or illness (Shaw 1993; Wanzer 1989). Inflammatory conditions result in increased glycogenolysis, protein catabolism, and fatty acid degradation, and insufficient nutrient intake causes depletion of organ proteins, ultimately leading to malnutrition, and increased risk of infection and death (Reintam 2017). Under these circumstances, enteral nutrition (EN) (enteral tube feeding) or parenteral nutrition (PN) (the delivery of calories and nutrients into a vein) can compensate for nutritional intake until oral intake becomes satisfactory (Bounoure 2016). Patients with reduced nutrient intake from the gastrointestinal tract have an increased risk of infection because of reduced gut integrity and the physiologic stress response (McClave 2009b).

Because of the better outcomes regarding infection, recent national guidelines backed by randomized controlled trials (RCTs) or systematic reviews, or both, have consistently suggested that EN should be used preferentially over PN in hospitalized patients who require non-oral nutrition therapy, excluding cases where EN is contraindicated (CCPG 2015; JSICM 2017; McClave 2016a; McClave 2016b; Reintam 2017). A recent Cochrane Review of all types of nutrition support in hospitalized patients reported that EN may alleviate serious adverse events (Feinberg 2017).

A large number of microorganisms exist in the gastrointestinal tract, and the gastrointestinal mucosa acts as a barrier against microbial infection. Furthermore, immune tissue known as the Peyer's patches located in the gastrointestinal mucosa plays a preventive role against bacterial contamination of the body (Reintam 2012). It has been proposed that when nutrients do not flow in the gastrointestinal tract, the gastrointestinal mucosa becomes atrophied, leaving the individual susceptible to infection due to the reduced interaction between the gut and the systemic immune response, and leads to poor prognosis in critically ill patients (McClave 2009b). In particular, for critically ill patients with highly variable metabolic and immune response to injury or illness, early nutrition intake via the intestinal tract, that is enteral feeding, is highly important. Many studies have suggested that in such patients, early enteral feeding reduces the rate of infection and mortality (McClave 2009b). Currently available clinical practice guidelines recommend that early EN should be administrated to patients, especially to those in the intensive care unit (ICU) (Reintam 2017).

Enteral feeding is a riskless, useful, and generally well-tolerated approach applied in patients with normal gastrointestinal (GI) tract and function (Zanetti 2016). The human GI tract has many roles, including digestion and absorption of nutrients and water, regulating the growth of intraluminal microorganisms, while maintaining barrier control, secretion, and endo/paracrine and immuno-logic functions. For proper GI functioning, perfusion, secretion, movement, and coordinated intestinal microbial interaction are essential (Reintam 2012).

In hospitalized patients, GI function is often impaired because of pre-existing disease (e.g. diabetes mellitus, vagotomy, myopathies); acute disease (e.g. shock, pancreatitis, spinal cord injury, trauma, abdominal surgery, burn); medication (e.g. sedatives, opioids, anticholinergics, vasopressors); or electrolyte abnormalities (e.g. hyperglycemia, hypokalemia) (Deane 2007; Zanetti 2016). The severity of existing disease is thought to be the main reason for GI dysfunction, and may influence the occurrence and degree of complications due to enteral nutrition (Deane 2007; Nguyen 2008); the sympathetic nervous system predominates over the parasympathetic nervous system, leading to reduced gastrointestinal peristalsis and reduced absorption capacity in the digestive tract, with a concomitant increase in enteral nutrient stagnation time in the stomach. Gastrointestinal dysfunction is a common event during critical illness, with an incidence rate of 63%, and can emerge as part of multi-organ failure (Montejo 1999; Reintam 2012).

Gastrointestinal dysfunction is often an obstacle to EN. Feeding intolerance (FI) signifies GI dysfunction, and manifests as a result of motility and absorption disorders of GI tract, frequently leading to reduced EN intake (Elke 2015; Zanetti 2016). Incidence of FI is about 27% in general ward patients, and about 36% in ICU patients (Gungabissoon 2015; Wang 2017). A systematic review of observational studies showed that in comparison with patients without FI, those with FI presented with higher infectious complications and ICU mortality, and longer ICU stays (Blaser 2014). The leading cause of FI is delayed gastric emptying. Gastric emptying can be assessed by various methods, such as scintigraphy, paracetamol absorption test, ultrasound, refractometry, breath test, and gastric impedance monitoring (Moreira 2009). In clinical practice, however, it is usually assessed by measuring the gastric residual volume (GRV). Gastric residual volume is the amount of liquid drained from a stomach following administration of enteral feed; this liquid consists mainly of infused nutritional formula or water, and secreted GI juice. Gastric residual volume is measured either by aspiration using a syringe, or by gravity drainage to a reservoir (Elke 2015). Though GRV can easily vary depending on the method of drainage, patient's position, amount of gastric juice, kind of tube (large/small diameter, pored/non-pored), and position of tube tip (Bartlett 2015; Metheny 2005), it is the preferred clinical indicator of gastric emptying because of its simplicity.

Monitoring GRV involves obtaining frequent GRV measurements and employing appropriate interventions in patients with large GRVs. Gastric residual volume monitoring (monitoring of residual volume of the enteral nutrients including digestive juices) is an essential component of EN patient care and aids in preventing complications due to EN (McClave 2009a; Metheny 2012). In healthy adults and patients with mild illness, seven to nine liters of digestive juices are secreted daily (Jeejeebhoy 1977; Jeejeebhoy 2002). Most of these juices are absorbed by the small bowel, while approximately 500 mL reaches the colon and 150 g remains in the stools. Administering additional enteral nutrients in patients with increased GRVs may cause aspiration and an increase in intra-abdominal pressure, which increases the risk of respiratory and circulatory failure, and intestinal necrosis. For this reason, it is particularly important to monitor GRV in the early stages of administration of enteral nutrition, especially in critically ill patients. Frequency of GRV measurement (e.g. every six hours) and the intervention strategy for large GRVs (e.g. if GRV is above 500 mL, hold feeding for two hours and re-check GRV) is usually decided as per institution-specific protocols and needs of the in-patient population (Bounoure 2016). Gastric residual volume is usually monitored in the ICU during nasogastric feeding or gastrostomy tube. Gastric residual volume monitoring is a well-established and common nursing practice in the ICU. Metheny reported that about 97.1% of critical care nurses reported GRV measurements in the United States (Metheny 2012). Optimal GRV monitoring involves standardization of several parameters, and the following aspects have been studied so far: the frequency of monitoring (Reignier 2013; Williams 2014), comparison of the methods of managing GRV to prevent complications (Booker 2000), and whether the remaining contents of the stomach should be returned to the stomach or discarded (Julien 2009; Williams 2010).

Feeding intolerance is highly prevalent and is associated with worse outcomes, especially in critically ill patients. It is thus imperative to standardize assessment of GI function and a bedside examination of the abdomen (JSICM 2017; McClave 2016a). Gastric residual volume monitoring is considered to be a simple and effective method of monitoring FI, and a recent systematic review showed that a large GRV was used to define FI in 83% of studies (Blaser 2014). The main purpose of monitoring GRV is to improve safety in patients receiving EN. The administration of more enteral nutrients via the feeding tube while the stomach is already full (a high GRV) is not advisable in patients with reduced GI tolerance. In such cases, the residual gastric fluid is refluxed from the stomach into the esophagus, causing vomiting and ultimately increasing the risk of aspiration pneumonitis caused by aspiration of the vomit (McClave 2009a). Aspiration pneumonitis is a severe complication that not only prolongs hospital stays but also increases mortality rates and the expense of hospitalization (Hayashi 2014). Hence, monitoring the GRV is considered an important procedure.

Gastric residual volume monitoring may enable clinicians to identify patients with delayed gastric emptying earlier, and deploy strategies to minimize the adverse effects of FI. The strategies to minimize FI include the use of prokinetic agents, postpyloric feeding, and pausing/decreasing EN (Elke 2015). Enteral nutrition protocols including interventions for cases with large GRVs may help achieve goal rate and prevent aspiration (Metheny 2010; Racco 2012). According to a national survey in the United States, almost 70% of critical care nurses used 200 mL or 250 mL as threshold levels for interrupting EN, and 80% of the nursing personnel measured GRV every four hours (Metheny 2012). However, apart from the aforementioned benefits, monitoring the GRV has several disadvantages (Edwards 2000). Gastric residual volume monitoring is unnecessary intervention because of the lack of evidence of efficacy of GRV monitoring in some cases and may result in an increase in the time taken to reach the target amount of enteral nutrients because of interrupted feeding (Edwards 2000).

Digestive juices are included in the residual contents of the stomach, and it is possible that important electrolytes and digestive enzymes are discarded along with the residual contents of the stomach, which might lead to electrolyte imbalances and poor digestion. Furthermore, GRV monitoring must be confirmed manually primarily by a nurse, and increases other costs of patient care as well (e.g. requirement of additional syringes). Unnecessary monitoring of the GRV therefore contrarily increases nursing burden, which may lower the quality of medical care.

Although GRV monitoring has been part of the recommendations for critical care for decades, the importance and clinical relevance of routine GRV monitoring has been frequently questioned. In recent years, GRV monitoring has been routinely performed as per institution-specific protocols in all patients receiving EN (Reintam 2017). However, as mentioned above, it has been suggested that routine monitoring of the GRV may increase the workload of nurses, thereby delaying treatment for other patients. Insufficient nursing care may also be a consequence of increased nursing burden. Furthermore, it is thought that the interruption of feeding for monitoring of the GRV increases time taken to reach the feeding goal, and also increases the risk of infection caused by malnutrition from insufficient energy intake and reduced usage of the gastrointestinal tract (Reintam 2017).

Recent RCTs of GRV monitoring may be classified into three categories: ‘higher versus lower GRV threshold,’ ‘not monitoring versus routine monitoring GRV,’ and ‘regular versus variable time interval of monitoring GRV.’ Two RCTs showed that increasing the threshold levels of GRV leading to pause/decrease of EN from 200 mL to 400 mL or 500 mL did not increase the occurrence of regurgitation, aspiration, or pneumonia (McClave 2005; Montejo 2010). Results from one RCT and one before-after study indicated that discontinuation of GRV monitoring improved delivery of EN without compromising patient safety (Poulard 2010; Reignier 2013). There were no significant differences in pneumonia incidence between GRV-monitored and GRV-unmonitored groups in these two trials. One RCT showed that a lower frequency of GRV monitoring was associated with more vomiting, but was not associated with worse outcomes including pneumonia (Williams 2014). Furthermore, GRV monitoring may result in tube clogging, improper discontinuation of EN, waste of medical resources including nursing time, and decreased supply of EN (Powell 1993).

Paralytic ileus (or adynamic ileus) is a condition in which there is a functional motor paralysis of the digestive tract secondary to neuromuscular failure involving the myenteric (Auerbach's) and submucous (Meissner's) plexus. The intestine fails to transmit peristaltic waves, resulting in a functional obstruction, and allowing fluid and gas to collect in the intestine. It is the small intestine that is predominantly affected, but the colon and stomach could also be involved. The resultant stasis leads to accumulation of fluid and gas within the bowel with associated distension, vomiting, decrease of bowel sounds, and absolute constipation. Paralytic ileus is a neurogenic condition, in which the normal electrical slow wave is present in the smooth muscle but does not excite any action potentials. The mechanism is thought to be adrenergic stimulation, possibly involving dopamine release, but adrenoceptor blockade or dopamine inhibition have not been shown to be therapeutically effective.^{1, 2} In postoperative ileus, it is the inhibitory alpha2-adrenergic reflexes with peptidergic afferents that contribute.² The motor inhibition is triggered by a variety of stimuli, the commonest being tactile stimulation during surgery and peritonitis. Paralytic ileus is commonly seen in postoperative ileus which resolves after 24-48 h or from hypokalemia with diuretic use.¹ The whole intestine could be affected and could become grossly distended. Small bowel motility returns within a few hours and gastric and colonic motility after a few days, depending on the degree of trauma.^{1, 3} It might be prolonged if there is hypokalemia, hypoproteinemia, or renal failure or if the bowel is allowed to get grossly distended.⁴ Uremia could also produce ileus, often with distention, vomiting, and hiccups.¹ Peritonitis (infection) causes ileus. Ileus is also sometimes associated with retroperitoneal trauma and hemorrhage, spine or rib fractures, severe trauma outside the abdomen, and the application of a plaster jacket. This occurs through a reflex sympathetic overstimulation. Paralytic ileus could also be classified on the basis of the site of hypomotility.

Acute gastric dilatation, which probably represents extreme gastric stasis from atonia following any abdominal surgical procedure, carries a high risk of aspiration and mortality. It can occur in patients with anorexia and other psychiatric conditions when they have been prescribed large doses of psychotropic drugs and with the gastric autonomic neuropathy of diabetes mellitus. Whilst remedied by insertion of a wide nasogastric tube, contributory electrolyte imbalances, particularly hypokalemia, must be corrected.¹ Acute colonic pseudo-obstruction (Ogilvie syndrome) results from colonic hypomotility and produces a massive but reversible dilatation of the colon. It occurs in critically ill or postoperative patients. It could carry a mortality rate as high as 45% if not recognized or colonoscopically or operatively decompressed following failure of conservative treatment, or laparotomy not carried out when clinical signs indicate cecal ischemia or perforation.⁵

Sequelae of Acute Mechanical Intestinal Obstruction

Intestinal obstruction could be mechanical, which presents with colicky pain from the increased peristalsis against the obstructive lesion, or paralytic, which is painless being aperistaltic. There is a degree of overlap as mechanical obstruction can progress to a paralytic obstruction (ileus). Obstruction of the bowel leads to bowel distension above the block, with increased secretion of fluid into the distended bowel. Bacterial contamination occurs in the distended stagnant bowel. Prolonged increase in intraluminal pressure impairs the viability of the bowel wall with subsequent diffusion of toxic bacterial products into the peritoneal cavity where they are absorbed and produce toxemia and death. Thus, there is a role for i.v. prophylactic antibiotics in ileus. A further complicating factor is potassium depletion, which leads to weakness and smooth muscle paresis, and could superimpose a state of adynamic ileus on the existing obstructive lesion. The consequences of intestinal obstruction are therefore a combination of progressive dehydration, electrolyte imbalance, and systemic toxicity due to migration of toxins and bacterial translocation either through the intact but ischemic bowel or through a perforation.

Localized Ileus

A localized ileus consists of a localized loop (sentinel loop) of dilated small bowel. The appearance is not diagnostic of intra-abdominal infection but rather an associated feature of a local irritation such as acute pancreatitis, with the ileus being in the same anatomical region as the pathology. Radiologically, there are one or two loops of small or large bowel with often air-fluid levels in the sentinel loops. In localized or generalized ileus there is air in the rectum or sigmoid colon unlike in mechanical small or large bowel obstruction.⁴

Generalized Peritonitis

Generalized peritonitis is a serious condition resulting from irritation of the peritoneum due to infection, such as perforated appendix, or from chemical irritation due to leakage of intestinal contents, such as perforated duodenal ulcer. In the latter case, superadded infection gradually occurs and Escherichia coli and Bacteroides are the commonest organisms. The peritoneal cavity becomes acutely inflamed with production of an inflammatory exudate that spreads throughout the peritoneum, leading to intestinal dilatation and paralytic ileus. Peritonitis causes ileus initially from the inflammation, second from the bacterial toxins, and finally the fibrinous adhesions produced could delay the return of bowel function. Bacterial lipopolysaccharide (LPS) causes ileus by initiating an inflammatory response within the intestinal smooth muscle layers and a subsequent decrease in in vitro and in vivo smooth muscle contractility.^{1, 2} The ileus in generalized peritonitis is characterized by multiple distended loops of bowel.¹⁻⁴

Postoperative Ileus

Although definitions vary considerably in the literature, postoperative ileus (POI) can be characterized by a temporary inhibition of gastrointestinal motility after surgical intervention due to non-mechanical causes that promote deficient oral intake. The paralytic state usually lasts from a few hours to 24 h in the small bowel, from 24 to 48 h in the stomach, and from 48 to 72 h in the colon.¹⁻⁴ The duration of POI correlates with the degree of surgical trauma and is most extensive (10-30%) following colon and rectal surgery.³ However, POI can develop after all types of surgery, including extraperitoneal surgery. The clinical consequences of postoperative paralytic ileus are profound, contributing to discomfort, increased catabolism because of hindered oral nutrition, immobilization, increased risk of pulmonary complications, and increased length of hospital stay.¹⁻⁴ Preliminary clinical studies on laparoscopic colon surgery have shown normalization of gastrointestinal function within 24-48 h, thereby shortening recovery and length of hospital stay.³ The pathophysiology of POI is multifactorial and complex involving pharmacological (opioids, anesthetics), with possible direct inhibition of enteric or spinal nerves, neural, and immune-mediated mechanisms.¹⁻⁴ The electrolyte imbalance following major surgery could contribute to paralytic ileus by interfering with the normal ionic shifts that occur during smooth muscle contraction.^{1, 3} The early neural phase, triggered by activation of afferent nerves during the surgical procedure, is short-term compared to the later inflammatory phase. The latter starts after 3-6 h and lasts several days, making it a more interesting target for treatment.⁶ In addition, there is need for the study of the potential alteration of neurotransmitter receptor activity within the enteric nervous plexus after manipulation of the bowel. The two insults, the manipulation of the small intestine and bacterial translocation, act synergistically to promote paralytic ileus.⁷ Gentle manipulation of the intestine leads to postoperative ileus through the promotion of a postoperative cellular inflammatory response within the manipulated intestinal smooth muscle layer.⁸ This is evident by visualizing the escape of fluorescent microspheres from the gut lumen in response to intestinal manipulation.³ Within a short time the endogenous luminal particles and bacterial products will activate or prime circulating leucocytes that eventually extravasate into the manipulated muscularis. This is in response to the upregulation of the intercellular adhesion molecule-1 on the endothelium of the muscularis vasculature and potentiates ileus.^{3, 9, 10} Postoperative ileus is known to favor intestinal bacterial overgrowth, a major potential morbidity factor in ileus.^{1, 9} Risk factors for development of postoperative paralytic ileus include increasing age, American Society of Anesthesiologists score of 3-4, open approach, operative difficulty, operation duration more than 3 h, bowel handling, drop in hematocrit, or need for a transfusion, increasing crystalloid administration, and delayed postoperative mobilization.^{1-4, 11} While treatment is expectant and supportive, significant investigations into strategies to mitigate development of POI or shorten the duration have been undertaken, with mixed results. However, there is significant evidence that a minimally invasive surgical approach and multimodal pain regimens, including epidural anesthesia/analgesia and opioid-free or opioid-reduced analgesia, reduce the development of POI.¹²⁻¹⁸ Chewing gum and early oral feeding postoperatively could stimulate gut motility but the data are not all conclusive.^{17, 19-22} In addition, early enteral nutrition reduces the infectious complications of ileus.^{3, 20} Early enteral nutrition is a component of the enhanced recovery after surgery protocol that decreases the development of POI in patients who undergo elective major gastrointestinal surgery, such as colorectal surgery.^{3, 20, 21}

Prolonged Postoperative Ileus

Short-lasting local or generalized ileus occurs with any abdominal operation. However, paralytic ileus could be prolonged if there is hypokalemia, hypoproteinemia, renal failure, a localized peritoneal sepsis, leakage of visceral contents such as pancreatic juice, intraperitoneal or retroperitoneal hematoma, or if the bowel is allowed to get grossly distended.^{3, 4, 22} Mechanical obstruction must be differentiated from paralytic ileus during the first 4-5 days, as the former might require surgical intervention.^{3, 4} Mechanical obstruction could be caused by pre-existing adhesions, new fibrinous adhesions, sepsis perhaps due to an intestinal leak or a hematoma, a technically inadequate anastomosis or stoma causing undue narrowing of the lumen, and an internal hernia or volvulus.²³⁻²⁵ After 5 days, the infective causes of obstruction, such as anastomotic leakage or other intra-abdominal collection, become more important in the differential diagnosis. If a limited disruption of a large bowel anastomosis is suspected, a careful gastrografin enema could confirm a leak.^{12, 26} Acute gastric dilatation is a complication of major upper abdominal surgery, commonly seen post-splenectomy. The presentation is subtle with persistent left shoulder tip pain, and hiccups as early warning signs. Unrecognized and untreated cases can have a fatal outcome due to vomiting and aspiration. Correction of biochemical abnormalities, such as potassium, is essential and some cases might require prokinetic drugs to help improve the motility.²⁷

Clinical Features of Paralytic Ileus

The signs of ileus are a cessation of any motor activity. There is usually no abdominal pain, certainly no colic, but possibly some tenderness from distension. There could be tachypnea because the diaphragms are pushed up and tachycardia from hypovolemia. Bowel sounds are absent, flatus is not passed, and there is consequent gastric stasis that could lead to hiccups, discomfort, and effortless vomiting, unless gastric aspiration has been carried out. The abdomen is distended and tympanic. The abdomen is usually silent on auscultation or quiet “tinkling” sounds due to gut distension are heard as the abdomen is moved.¹⁻⁴ In pseudo-obstruction, the motility disorder commonly affects the whole gastrointestinal tract, although it is colonic distension that is often most prominent.⁵ Dilatation of the colon with signs of systemic upset (“toxic dilatation”) is becoming less common as acute attacks of ulcerative colitis are recognized and appropriately treated. Clinical appearance can be deceptive due to steroids; the danger if surgery is inappropriately delayed is colonic perforation, which still carries a high mortality. Colitis can also occur in Crohn's and rarely in ischemic or infective colitis (e.g., Yersinia enterocolitica, Campylobacter sp., and pseudomembranous colitis due to Clostridium difficile).²⁸ The radiological appearance of paralytic ileus is diagnostic. A plain X-ray of the abdomen in the upright posture shows dilated loops of bowel with multiple fluid levels, indicating distension with fluid and air in all of the small bowel and often much or all of the large bowel.^{3, 6} An abdominal and pelvic computed tomography (CT) scan is used to confirm the diagnosis of postoperative ileus in cases when X-ray is not diagnostic. Findings on CT scan diagnostic of POI include multiple air-fluid levels throughout the abdomen, elevated diaphragm, dilatation of both large and small intestine, and no evidence of mechanical obstruction. Computed tomography scan with i.v. contrast and oral water soluble contrast can also distinguish early postoperative ileus from mechanical obstruction.²⁹

Treatment of Paralytic Ileus

Preventative measures include avoiding unnecessary exposure and excessive handling of the bowel or traction on its mesentery.⁴ Treatment is conservative, as the condition is mostly self-limiting. Recovery is hastened by the correction of any fluid or electrolyte deficits by giving fluid and nutrients parenterally, and by measures to “defunction” the small bowel by nasogastric decompression. Hypokalemia especially needs attention. Fluids and food by mouth are withheld until bowel sounds return or flatus passed. The cause of the ileus should be found and dealt with. Conservative treatment often succeeds in postoperative ileus and contractility could return, particularly if electrolyte and fluid balance can be restored. Recently, several studies on nasogastric intubation questioned its efficacy in upper gastrointestinal surgery.³⁰⁻³² The insertion of a nasogastric tube could postpone the return of bowel sounds and increase the incidence of nausea and patient discomfort, but it does not affect the incidence of postoperative ileus.³⁰ The fluid lost by intubation is predominantly alkaline and this leads to acidosis. Parenteral infusion of normal saline should thus be supplemented by lactate or carbonate or by a balanced salt solution (Ringer's or Hartmann's solution). It is important to remember while calculating the daily fluid balance that these patients will need fluid to replace not only the ongoing losses (e.g., nasogastric aspirates) and baseline daily requirements but also the long-standing fluid deficits, or third-space loss. The patients with long-standing fluid losses could be deficient of 5 L or more of extracellular fluid, which should be replaced slowly over the first 48 h to prevent overexpansion of the circulating plasma volume. Drugs have little place, but prokinetic drugs such as cisapride have been used parenterally to stimulate peristalsis. Trials of adrenergic blockade combined with cholinergic stimulation have met with little success.^{2, 3, 33} The return of function is heralded by decreasing aspirates and abdominal girth, and the passage of flatus. If ileus is associated with a mechanical cause such as intra-abdominal sepsis, strangulated obstruction, or peritonitis, the relief of distension by intubation and rapid resuscitation are critically important prior to immediate surgical intervention.^{3, 6, 7, 33} It is physiologically important that a patient should not remain obstructed for more than 48 h as both the ensuing local bowel and systemic complications would worsen the prognosis.³

Conventional method of estimating distance are cumbersome, require large components and can only be performed in certain specific and dedicated examination areas, limiting continuous monitoring, causing delays in diagnosis and limiting value for prognostication. What is needed in the art is a device, system and method for continuous estimation of organ and tissue size and content within an intact organism.

### SUMMARY OF THE INVENTION

In one embodiment, an internal estimation system includes a flexible tube having a first resonator, the flexible tube configured to advance into an anatomical lumen of a subject, a second resonator configured to attach to a skin surface of the subject, wherein the first and second resonator are resonantly coupled, and a controller connected to the first resonator and the second resonator, wherein the controller is configured to estimate a distance between the first and second resonator based on an electrical signal parameter measured by the first resonator. In one embodiment, the electrical signal parameter is signal amplitude. In one embodiment, the electrical signal parameter is signal frequency. In one embodiment, the electrical signal parameter is signal quality. In one embodiment, the controller is configured to estimate a parameter of content within the anatomical lumen based on the electrical signal parameter. In one embodiment, the content is one of air, liquid and solid contents. In one embodiment, the first resonator is one of a plurality of resonators attached to the flexible tube. In one embodiment, the second resonator is one of a plurality of resonators attached to the skin surface of the subject. In one embodiment, the first resonator has a spiral geometry. In one embodiment, the first resonator is attached to an outer wall of the flexible tube. In one embodiment, the second resonator is planar. In one embodiment, the second resonator has a radially disposed perimeter comprising a plurality of alternating concave and convex curves. In one embodiment, at least one of the first and second resonator comprises a copper wire winding. In one embodiment, at least one of the first and second resonator comprises a polyamide surface layer.

In one embodiment, a method for internal estimation includes the steps of advancing a flexible tube having a first resonator into an anatomical lumen of a subject, attaching a second resonator to a skin surface of the subject, wherein the first and second resonator are resonantly coupled, and estimating a distance between the first and second resonator based on an electrical signal parameter measured by the first resonator. In one embodiment, the electrical signal parameter is signal amplitude. In one embodiment, the electrical signal parameter is signal frequency. In one embodiment, the electrical signal parameter is signal quality. In one embodiment, the method includes the step of estimating a parameter of content within the anatomical lumen based on the electrical signal parameter. In one embodiment, the content is one of air, liquid and solid contents. In one embodiment, the first resonator is one of a plurality of resonators attached to the flexible tube. In one embodiment, the second resonator is one of a plurality of resonators attached to the skin surface of the subject. In one embodiment, the first resonator has a spiral geometry. In one embodiment, the first resonator is attached to an outer wall of the flexible tube. In one embodiment, the second resonator is planar. In one embodiment, the second resonator has a radially disposed perimeter comprising a plurality of alternating concave and convex curves. In one embodiment, at least one of the first and second resonator has a copper wire winding. In one embodiment, at least one of the first and second resonator has a polyamide surface layer.

In one embodiment, an estimation system includes multiple resonantly coupled resonators configured to attach to a skin surface of the subject, and a controller connected to the plurality of resonantly coupled resonators and configured to estimate a distance between at least a first and second resonantly coupled resonator of the plurality of resonantly coupled resonators based on an electrical signal parameter measured by the first resonantly coupled resonator. In one embodiment, the electrical signal parameter is signal amplitude. In one embodiment, the electrical signal parameter is signal frequency. In one embodiment, the electrical signal parameter is signal quality. In one embodiment, the controller is configured to estimate a parameter of content within the anatomical lumen based on the electrical signal parameter. In one embodiment, the content is one of air, liquid and solid contents. In one embodiment, the second resonator is planar. In one embodiment, the second resonator has a radially disposed perimeter comprising a plurality of alternating concave and convex curves.

A method for estimation includes the steps of attaching multiple resonantly coupled resonators to a skin surface of the subject, and estimating a distance between at least a first and second resonantly coupled resonator of the plurality of resonantly coupled resonators based on an electrical signal parameter measured by the first resonantly coupled resonator. In one embodiment, the electrical signal parameter is signal amplitude. In one embodiment, the electrical signal parameter is signal frequency. In one embodiment, the electrical signal parameter is signal quality. In one embodiment, the method includes the steps of estimating a parameter of content within the anatomical lumen based on the electrical signal parameter. In one embodiment, the content is one of air, liquid and solid contents. In one embodiment, the second resonator is planar. In one embodiment, the second resonator has a radially disposed perimeter comprising a plurality of alternating concave and convex curves.

In one embodiment, an estimation system includes a controller connected to a plurality of resonantly coupled resonators, the controller configured to estimate a multi-dimensional geometry based on an electrical signal parameter measured by plurality of resonantly coupled resonators. In one embodiment, the multi-dimensional geometry includes a vertical and horizontal component. In one embodiment, the multi-dimensional geometry includes an X, Y and Z dimension. In one embodiment, the electrical signal parameter is indicative of organ or tissue size, or at least one of air, water, fluid, blood, CSF, urine, feces, pus and foreign material.

In one embodiment, an estimation method includes the steps of positioning multiple resonantly coupled resonators near an anatomical structure of the patient and estimating a multi-dimensional geometry based on an electrical signal parameter measured by the plurality of resonantly coupled resonators. In one embodiment, the multi-dimensional geometry includes a vertical and horizontal component. In one embodiment, the multi-dimensional geometry includes an X, Y and Z dimension. In one embodiment, the electrical signal parameter is indicative of at least one of air, water, fluid, blood, CSF, urine, feces, pus and foreign material.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A is a functional diagram of an internal estimation system according to one embodiment, FIG. 1B is an equivalent circuit diagram of a magnetic resonant coupling system according to one embodiment, FIG. 1C is a diagram of skin and tube sensor geometries according to one embodiment, FIG. 1D is a table of optimized sensor geometry specifications at 13.56 MHz according to one embodiment, FIG. 1E is a functional diagram of an internal estimation system according to one embodiment, where the first resonator is one of a plurality of resonators attached to the flexible tube, and FIG. 1F is a functional diagram of an internal estimation system according to one embodiment, where the second resonator is one of a plurality of resonators attached to the skin surface of the subject.

FIGS. 2A, 2B and 2C are graphs according to one embodiment of S21 vs. distance vs. frequency in skin and tube-1, skin and tube-2, and skin and tube-3 sensors according to one embodiment.

FIGS. 3A and 3B according to one embodiment show an experimental setup in saline solution (FIG. 3A) and measured transmission coefficient at 100 mm distance for skin and tube-2 sensors (FIG. 3B).

FIGS. 4A, 4B and 4C according to one embodiment show prediction results in a blood medium, showing graphing distance prediction and their residual plots for skin and tube-1, skin and tube-2, and skin and tube-3 sensors.

FIGS. 5A, 5B and 5C according to one embodiment show prediction results in a saline medium, graphing distance prediction and their residual plots for skin and tube-1, skin and tube-2, skin and tube-3 sensors.

FIGS. 6A, 6B and 6C show according to one embodiment a 30 days drift test, graphing transmission coefficient vs. distance for skin and tube-1, skin and tube-2, skin and tube-3 sensors.

FIG. 7 shows graphs of experimental results in air, saline and blood mediums between one right and three different left resonators according to one embodiment.

FIG. 8 shows an experimental setup and graphical results for a prediction of water content in biological tissue according to one embodiment.

FIGS. 9A and 9B show a diagram and calculations for estimation of NaCl and H2O fractions according to one embodiment.

FIGS. 10A, 10B and 10C show diagrams of a system embodiment where resonators are applied to the abdominal wall without a feeding tube inside the patient according to one embodiment.

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in devices, systems and methods for estimation of organ and tissue size and content. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a device, system and method for continuous estimation of organ and tissue size and content within an intact organism.

Electrical current comprises of waves of energy which decay away from the source with a mathematical precision dependent on the medium it is present (air, liquid, solid and semi-solid medium). Two resonantly coupled sensors at same frequency allows for a small electrical current to be passed from one to other and its strength to be detected. The decay in the electrical signal is like a stone dropped in the pond of water with the strongest waves close to where the stone was dropped and then reducing in amplitude of the waves of water as the distance from the original site of stone away from it. Electromagnetic principles allow fairly accurate estimation with 98% accuracy in this regimen.

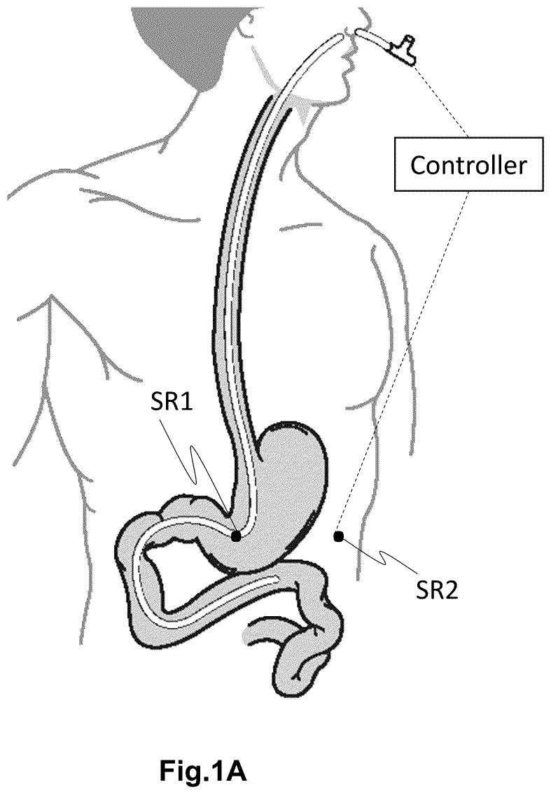

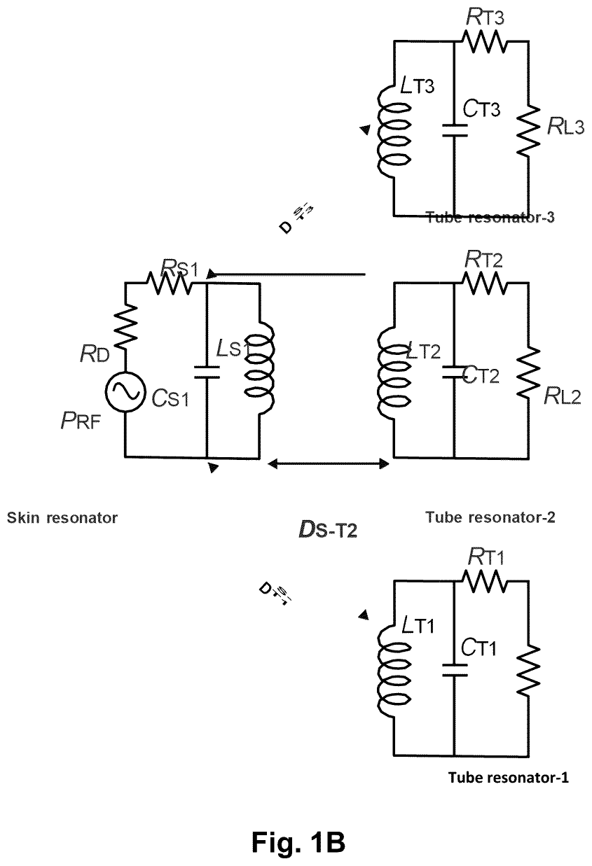

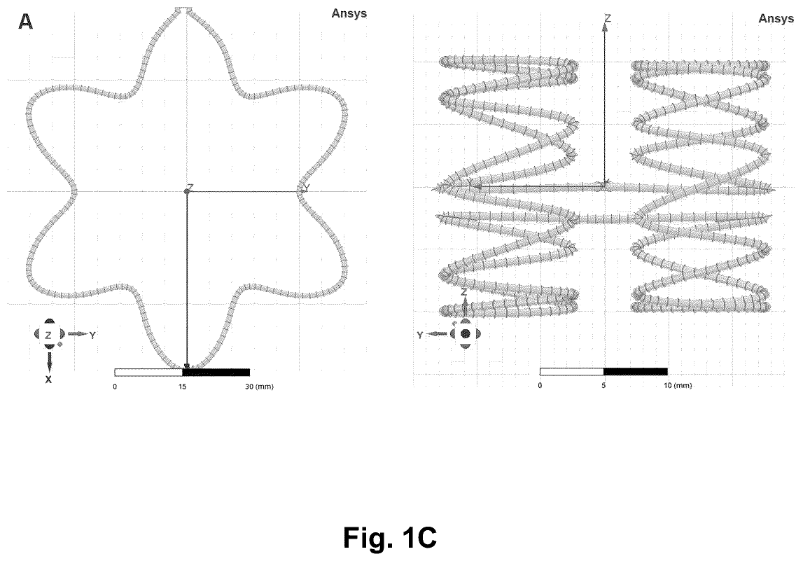

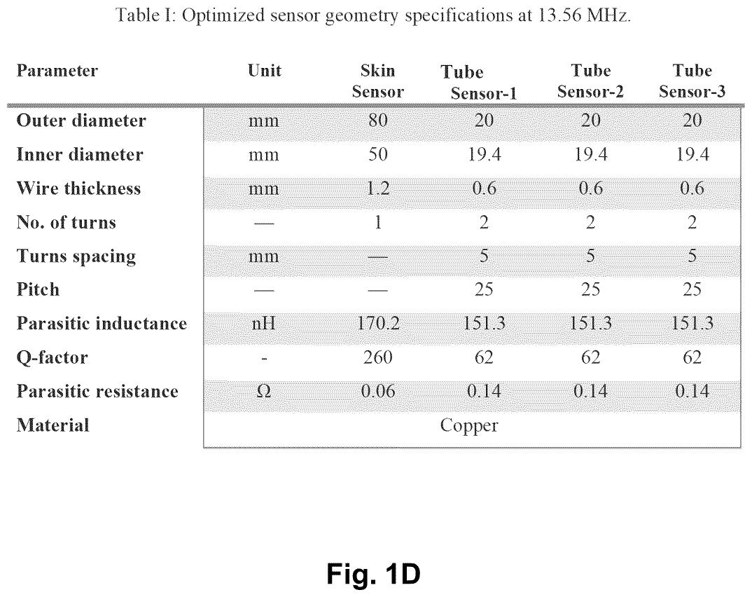

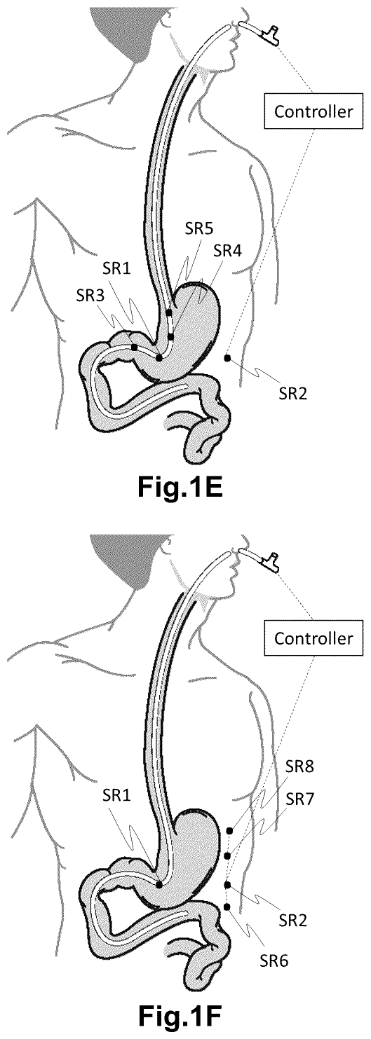

For example and with reference to FIG. 1A, if one were to position a sensing resonator SR1 on the feeding tube inserted in the stomach or intestine and a sensing resonator SR2 were to be placed on the abdominal wall, the distance between the two can be accurately predicted based on the calculated distance between the two resonators. Similarly, the distance and quality of signal will be different for air, liquid and solid contents within the stomach or intestine.

In another embodiment, the resonators placed on either side of the abdomen can give similar distance estimation and content estimation of the abdominal cavity which can help detect, predict and monitor dilatation of intestinal loops and stomach which happens after abdominal surgery. The resonators can be flexible and can be incorporated in the regular dressing that is applied or can be modified to have multiple components such as ECG electrodes placed on skin of the abdominal wall. Multiple resonators can be used both internally and on the surface of the skin.

With reference to FIG. 1B, an equivalent circuit model of a magnetic resonant coupling system is shown according to one embodiment. This system uses one high Q resonator on the transmitter side and three high Q resonators on the receiver side. L_{A}, L_{O1}, L_{O2}, and L_{O3} are parasitic inductances, R_{A}, R_{O1}, R_{O2}, and R_{O3} are parasitic resistances, and C_{A}, C_{O1}, C_{O2}, and C_{O3} are tuning capacitances. R_{S} is source resistance and R_{L1}, R_{L2}, and R_{L3} are load resistances. D_{A-O1}, D_{A-O2}, and D_{A-O3} are the distance between the sensors. The sensor is driven by the applied RF power (P_{RF}), and power is then wirelessly transmitted to other sensors through magnetic coupling. The size and shape of the sensors can have a significant impact on the range, measurement on degrees of freedom, and efficiency. Example skin and tube sensor geometries are shown in FIG. 1C according to one embodiment. ANSYS Electronics Desktop 2021 R2 can be used to optimize the resonators in terms of turns, outer diameter, wire thickness, wire spacing, and pitch at an operating frequency of 13.56 MHz (see Table 1, FIG. 1D). A copper wire can be used for winding and polyamide as the top layer to make it biocompatible. The Q-factor of resonators is obtained using Qᵢ=Rᵢ/ωₒLᵢ, where ωₒ=2 πf. The parasitic inductance and resistances are obtained using Lᵢ=Im(Zᵢᵢ)/ωₒ and Rᵢ=Re(Zᵢᵢ)^{[18]}.

Accordingly, in one embodiment, an internal estimation system can include a flexible tube with a first resonator, while a second resonator attaches to a skin surface of the subject. The first and second resonator are resonantly coupled. A controller is connected to the first and second resonator and configured to estimate a distance between the resonators based on an electrical signal parameter measured by the first resonator. The electrical signal parameter can be for example amplitude, frequency and/or signal quality. The controller can estimate a parameter of content within the anatomical lumen based on the electrical signal parameter (for example, air, liquid and solid contents). The first resonator can be one of multiple of resonators attached to the flexible tube (FIG. 1E), while the second resonator can be one of a plurality of resonators attached to the skin surface of the subject (FIG. 1F). In one embodiment, an estimation system includes multiple resonantly coupled resonators configured to attach to a skin surface of the subject and a controller configured to estimate a distance between at least a first and second resonantly coupled resonator based on a measured electrical signal parameter.

In one embodiment, an estimation system includes multiple resonantly coupled resonators and a controller connected to resonators and configured to estimate a multi-dimensional geometry based on electrical signal parameters measured by the resonators. The multi-dimensional geometry may include a vertical and horizontal component, or an X, Y and Z dimension. The electrical signal parameter may be indicative of an organ or tissue size, or at least one of air, water, fluid, blood, CSF, urine, feces, pus and foreign material. The various embodiments will be explained in further detail below with reference to the experimental examples.

In one embodiment, a method for internal estimation includes the steps of advancing a flexible tube having a first resonator into an anatomical lumen of a subject, attaching a second resonator to a skin surface of the subject, wherein the first and second resonator are resonantly coupled, and estimating a distance between the first and second resonator based on an electrical signal parameter measured by the first resonator. In one embodiment, the electrical signal parameter is signal amplitude. In one embodiment, the electrical signal parameter is signal frequency. In one embodiment, the electrical signal parameter is signal quality. In one embodiment, the method includes the step of estimating a parameter of content within the anatomical lumen based on the electrical signal parameter. In one embodiment, the content is one of air, liquid and solid contents. In one embodiment, the first resonator is one of a plurality of resonators attached to the flexible tube. In one embodiment, the second resonator is one of a plurality of resonators attached to the skin surface of the subject. In one embodiment, the first resonator has a spiral geometry. In one embodiment, the first resonator is attached to an outer wall of the flexible tube. In one embodiment, the second resonator is planar. In one embodiment, the second resonator has a radially disposed perimeter comprising a plurality of alternating concave and convex curves. In one embodiment, at least one of the first and second resonator has a copper wire winding. In one embodiment, at least one of the first and second resonator has a polyamide surface layer.

### Experimental Examples

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

Simulation Results

Three positions for the sensors were examined while the receive sensor remains affixed to skin outside. The three locations for the tube sensors are medial, inferior, and superior to the location of the skin sensor. A tilting angle of 30° was chosen for skin, 15° for tube-1, 10° for tube-2, and 15° for tube-3 sensors based on the average angle produced by the skin and the feeding tube within the stomach. The skin and tube sensors in blood medium (permittivity (εr) of 210.64, loss tangent (tan δ) of 7.0297, and conductivity (S/m) of 1.117) used in the simulation study. The maximum Q-factor of skin sensor and tube sensor were 260 and 62, and the maximum self-inductance values were 170.2 nH and 151.3 nH, respectively. The parasitic resistance of the skin and tube sensors was 0.06Ω and 0.14Ω. To validate the transmission coefficient (S21), the sensor's resonant frequency was obtained using fᵣ=½π·sqrt(LC), where fᵣ is the resonant frequency, L is the parasitic inductance, and C is the tuning capacitance. C_{A}, C_{O1}, C_{O2}, and C_{O3} were found to be 0.8 nF, 0.91 nF, 0.91 nF, and 0.91 nF in ANSYS Circuit software. The impedances of the source and load were set to 50Ω.

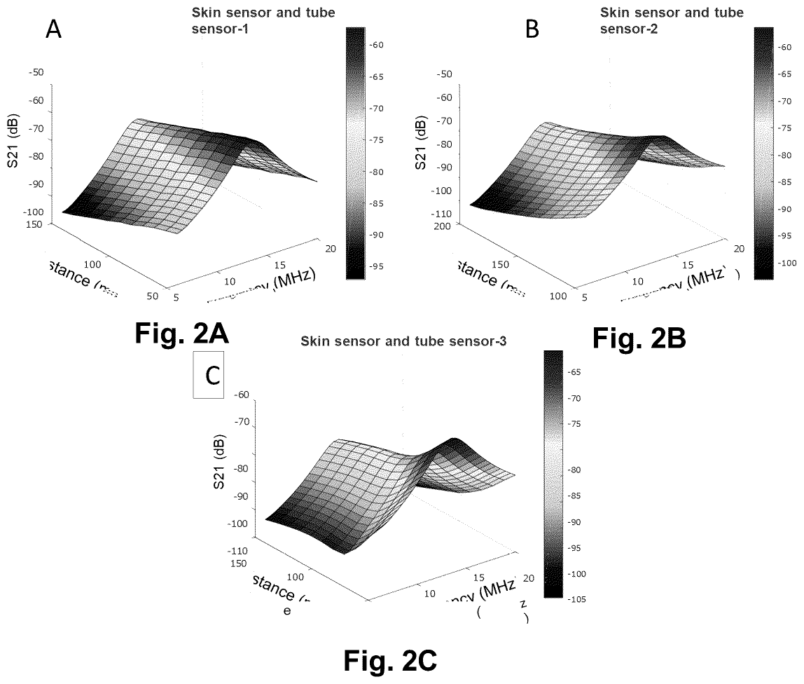

Three sets of simulations were performed skin and tube-1, skin and tube-2, and skin and tube-3 sensors, respectively. In the first simulation, tube-1 sensor at 50 mm negative y-axis was considered with respect to skin sensor and varied distance from 50 mm to 150 mm with a 10 mm step size along the z-axis. In the second simulation, tube-2 sensor was positioned parallel to skin sensor and varied the distance from 100 mm to 200 mm with a 10 mm step size along the z-axis. In the final simulation, tube-3 sensor was set to a 75 mm positive y-axis with respect to skin sensor and varied the distance along the z-axis from 50 mm to 150 mm with a 10 mm step size. These ranges are meant to account for anatomical variation between stomach and skin. The simulated S21 vs. distance vs. resonant frequency of skin and tube-1, skin and tube-2, and skin and tube-3 sensors are shown in FIGS. 2A, B, and C. The results show that as the distance between the sensors increases, S21 decreases exponentially.

Measurement and Prediction Results

The optimized sensors found in ANSYS Electronics Desktop 2021 R2 were fabricated using polyamide outer coating copper wire. The tube sensors are positioned inferior, medial, and superior to the skin sensor, respectively, shows the fabricated skin sensor in a radial fashion. For precise measurements, an acrylic box was created with a volume of 320 mm×320 mm×320 mm (width×length×height) based on the sensors' maximum separation distance using Universal Laser System (Scottsdale, Arizona). Four acrylic holders were created for positioning skin and tube sensors in the x, y, and z directions.

Transmission Coefficient (S21)

To adjust the resonance frequency of sensors NanoVNA-H was used. The skin sensor was connected to port-1 of NanoVNA-H and tuning was started at a capacitance of 1 nF, which was chosen as a starting point because the simulation produced the desired resonant frequency at 0.8 nF. At 1 nF, the resonant frequency was measured to be 13 MHz. The capacitance was decreased in step sizes of 0.1 nF until 0.60 nF, at which the resonant frequency was near the operating frequency. By serial measurements, 0.69 nF was found to be the optimal capacitance to achieve the resonant frequency of 13.56 MHz. The same tuning procedure was repeated for tube-1, tube-2, and tube-3 sensors and the optimal capacitance value was found to be 0.78 nF. Using the equation mentioned in section II, the self-inductance of the skin and tube sensors was found to be 199.6 nH and 176.6 nH, respectively.

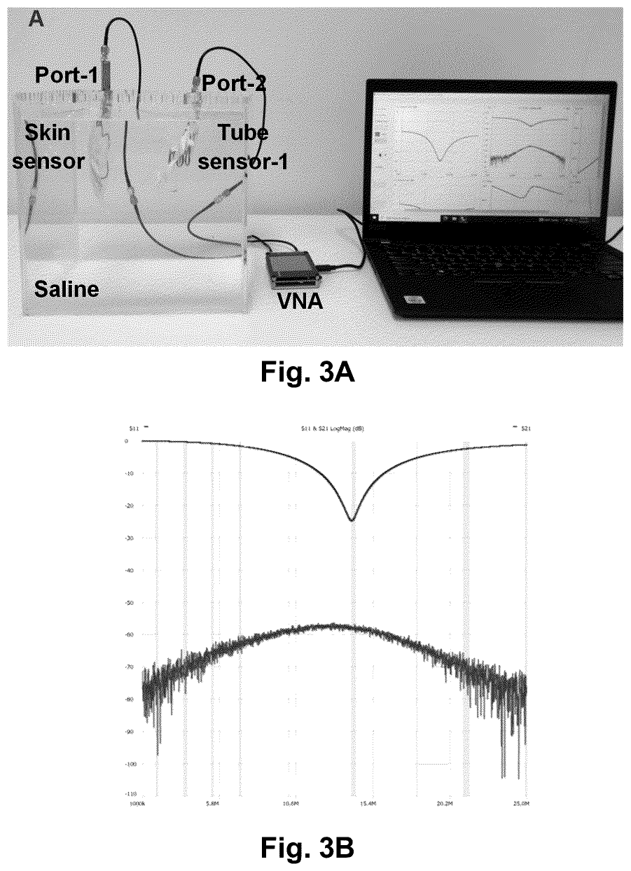

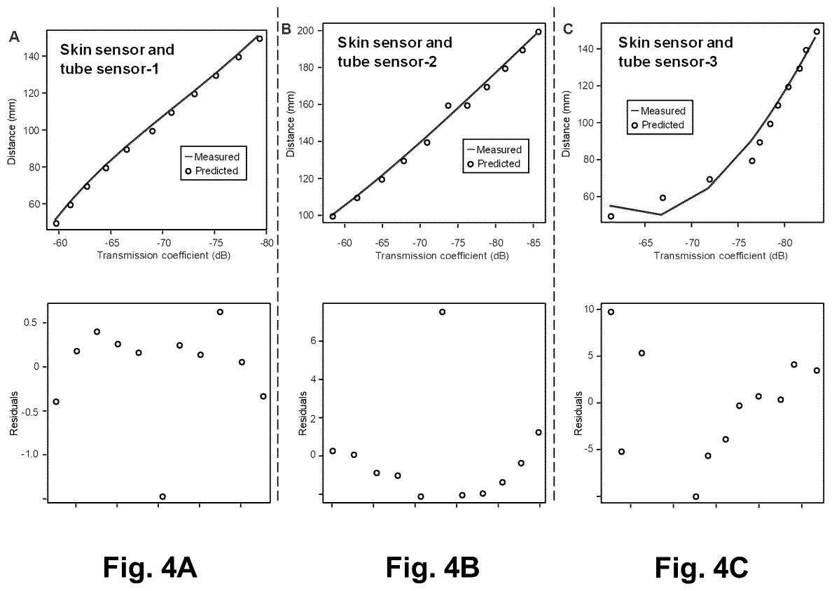

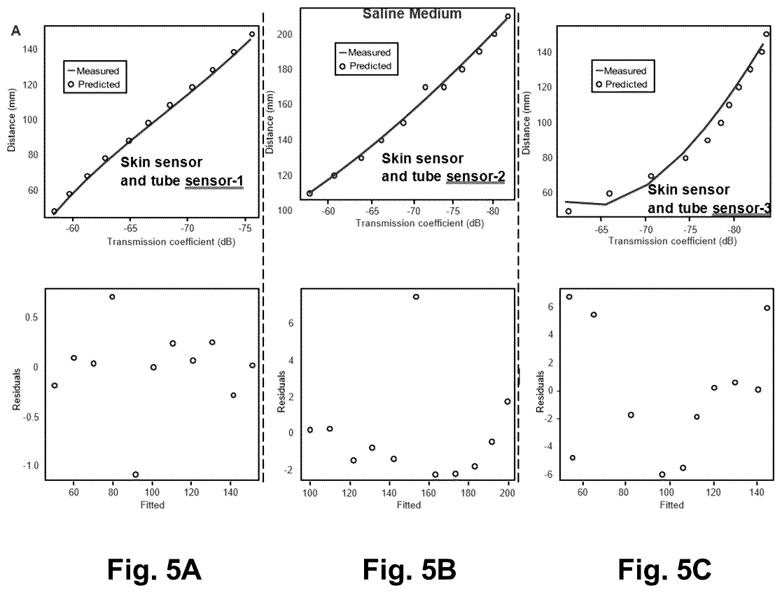

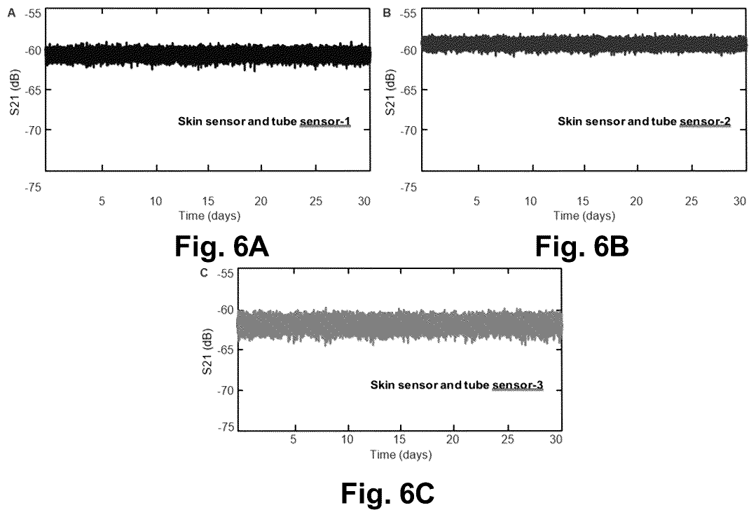

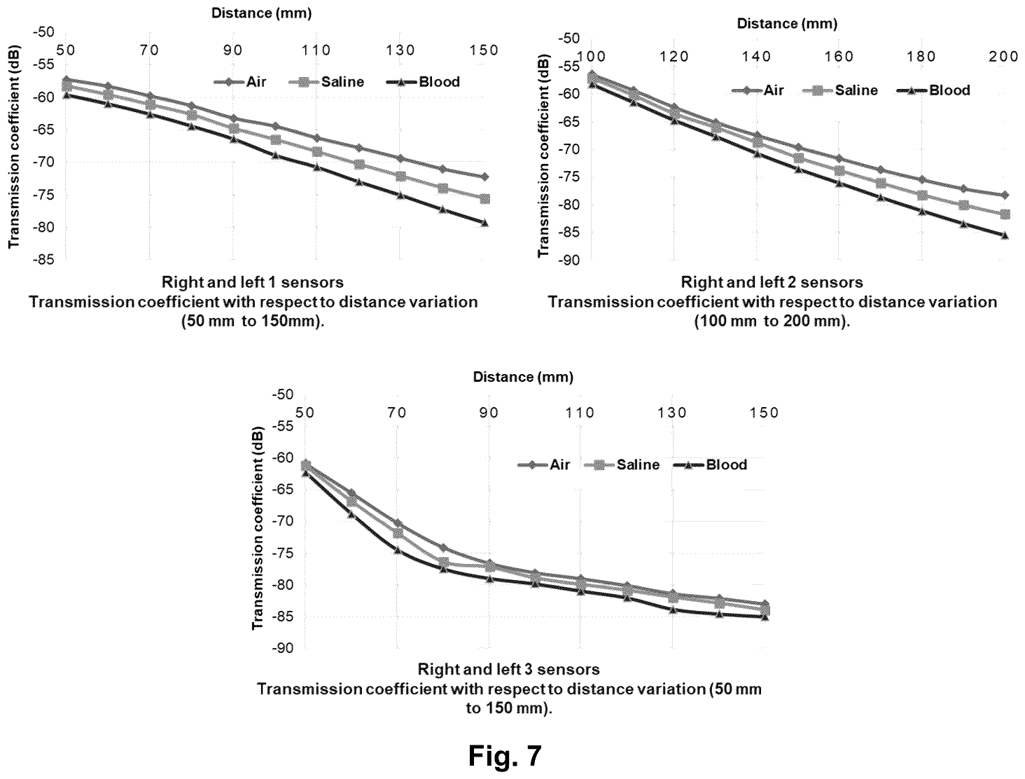

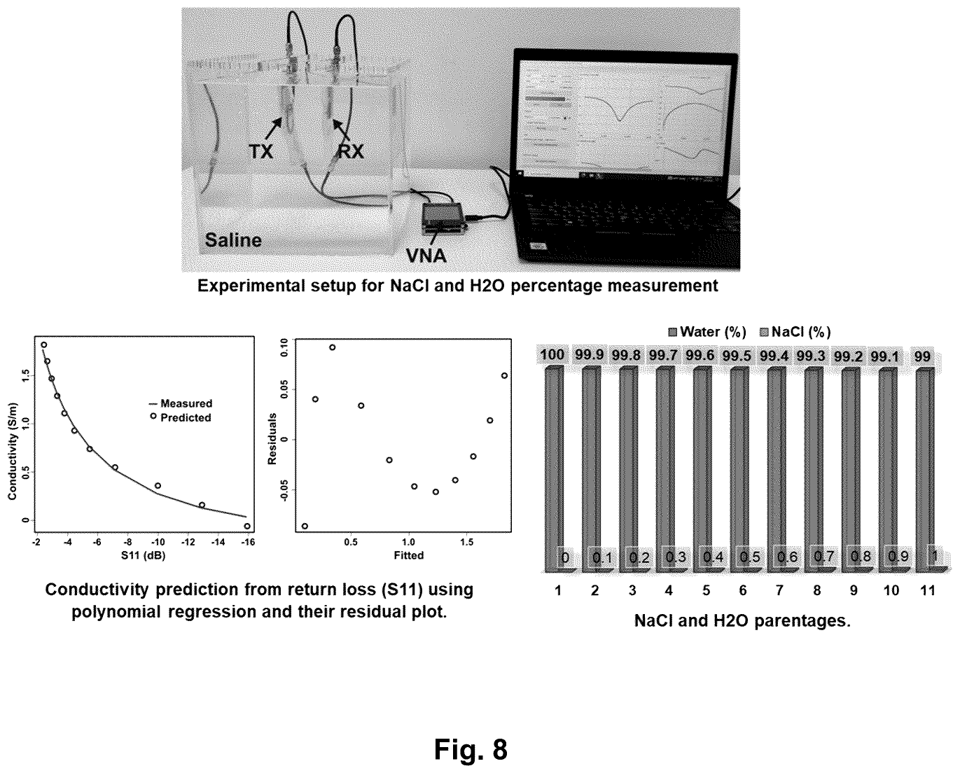

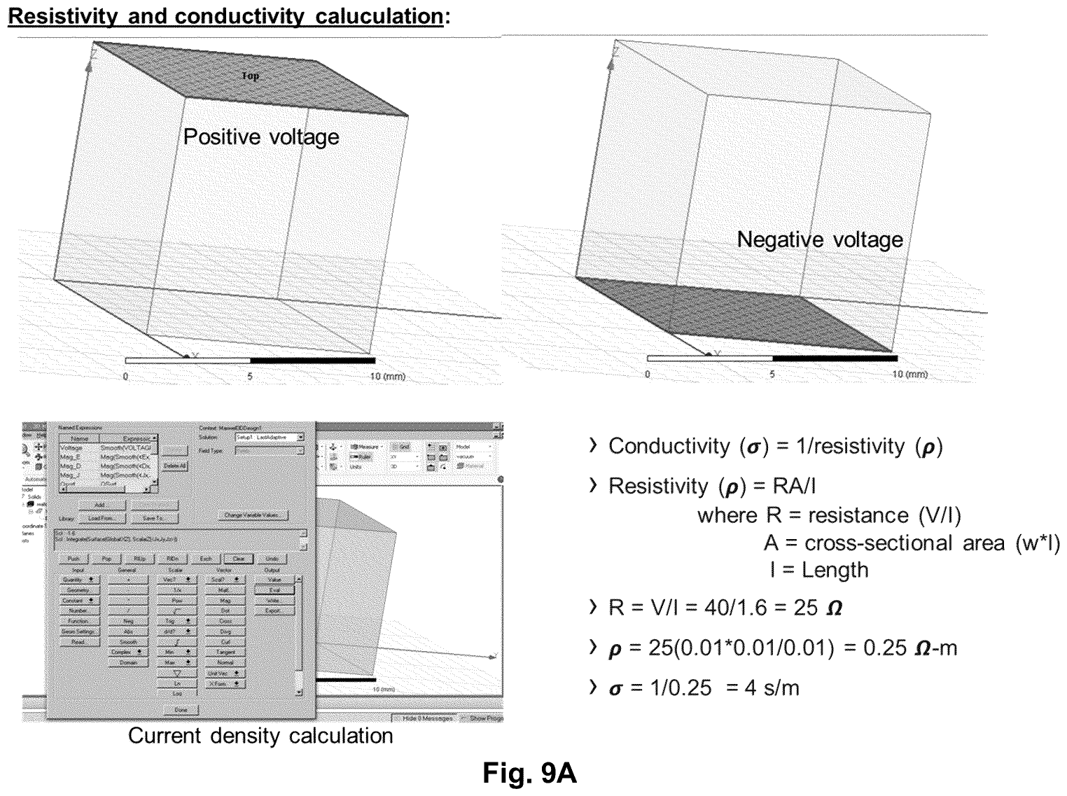

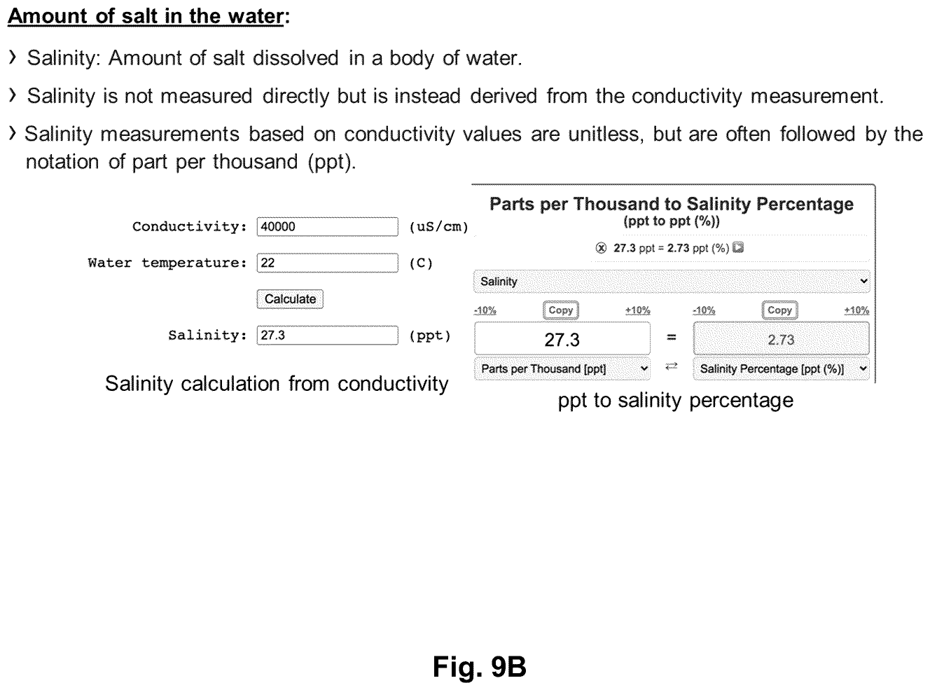

As shown in FIG. 3A, the sensors were immersed in porcine blood and saline solution using acrylic holders to measure the S21. Firstly, the skin sensor was connected to port-1 and the tube-1 sensor was connected to port-2 of NanoVNA-H, forming a two-port configuration with both ports having a default impedance of 50Ω. S21 was measured by systematically varying the distance between the skin and tube-1 sensor along the y-axis. The same procedure was repeated to measure the S21 between skin and tube-2 sensors, and skin and tube-3 sensors. The measured results indicate that the S21 decreases exponentially as the distance between the sensors increases. The measured transmission coefficient for skin and tube-1 sensors was −59.61 dB at 50 mm negative y-axis and 50 mm positive z-axis distance, −58.21 dB at 100 mm positive z-axis distance for skin and tube-2 sensors, and −61.2 dB at 75 mm positive y-axis and 50 mm positive z-axis distance for skin and tube-3 sensors with porcine heart. The measured transmission coefficient for skin and tube-2 sensors is shown in FIG. 3B. S21 vs. distance between the sensors with porcine blood is plotted in FIGS. 4A, B, and C, respectively. The measured transmission coefficient for sensors with saline solution is plotted in FIGS. 5A, B, and C. To examine drift, continuous measurements were taken between 13.5 MHz and 13.6 MHz resonant frequency. The millions of sampling cycles were analyzed using MATALAB. At a distance of 50 mm, the minimum and maximum transmission coefficients for skin and tube-1 sensors are −59.24 dB and −60.06 dB, respectively. The minimum and maximum transmission coefficients for skin and tube-2 sensors at 100 mm distance are −57.91 dB and −58.95 dB, respectively. The minimum and maximum transmission coefficients for skin and tube-3 sensors at 50 mm distance are −60.63 dB and −62.15 dB, respectively. The sensors were proven to run continuously for 30 days without any interruptions and the results are shown in FIGS. 6A, B, and C.

Distance Prediction

R studio version 1.1.463 was used to create a model to predict the distance from the skin to the three sensors on the tube as a function of the transmission coefficient. Due to the nature of our data, exponential regression models were considered first, however, a polynomial regression with log-transformed x values was found to have the best fit for all three sensor locations.

y=a−b·log(x)+c·log(x)2−d·log(x)3 Model:

For skin and tube-1, and skin and tube-3 sensors, a minimum and maximum distance of 50 mm and 150 mm were considered, with incremental distances of 10 mm. For skin and tube-2 sensors, a minimum and maximum distance of 100 mm and 200 mm were considered, with increments of 10 mm.

A log transformation was applied to the x data points because this was found to improve the fit of the model as well as the stochasticity of the residual plots. The polynomial model was observed to have adjusted R squared values of 0.9996, 0.9904, and 0.9628 for tube-1, tube-2, and tube-3 sensors, respectively, as well as p-values less than 0.05 for each coefficient, indicating that the model was a good fit for the data.

The residuals of our polynomial model were plotted to demonstrate that there were no observable patterns in the residuals and that the residuals were approximately normally distributed about zero. Both observations further vindicated this model's predictive utility. For tube-2 sensor, however, there was a slight pattern observed about zero in the residual plot, indicating that there may be room for improvement in the model used for distance prediction for the middle sensor. The residual standard errors (RSE) for tube-1, tube-2, and tube-3 sensors were 0.676, 3.264, and 6.398, respectively. The reasonable RSE for tube-2 sensor contrasted the conclusion drawn from the pattern observed in the residual plot and provided indication that the model may indeed not be overfitted and could be used to accurately predict distance to the middle sensor on the tube from the skin.

A common and intuitive measure of the accuracy of a prediction model is mean absolute percentage error. The polynomial regression model predicted the distance from the skin to the inferior, medial, and superior sensors on the tube with a mean absolute percentage error of 0.007, 0.010, and 0.057, respectively. In other words, the model was, on average, off-target by 0.77-5.75%.

The proposed sensors in this investigation have shown a high degree of precision with minimal drift data at a short to mid-term duration. Additionally, drift in these sensors can be addressable by automatically tuned capacitors at the fabrication stage. The power needed is miniscule for operation and the control mechanisms are easy to incorporate in existing feeding tubes without unduly increasing the controller size. Given the exclusive relationship between resonance that is related to a specific frequency band, our extensive investigations in terms of interference has shown that unless a material that has the same resonant frequency as the two sensors, it will not impede the signal.

Incorporating auto-tuning based operation the issues of sensor drift that plagues most of the implantable sensors will address. Additionally, the clinical practice of imaging (Xray), which allows for triangulation and calibration of the distances between the skin and tube sensor. The periodic calibration of distances will help with accuracy on an ongoing basis.

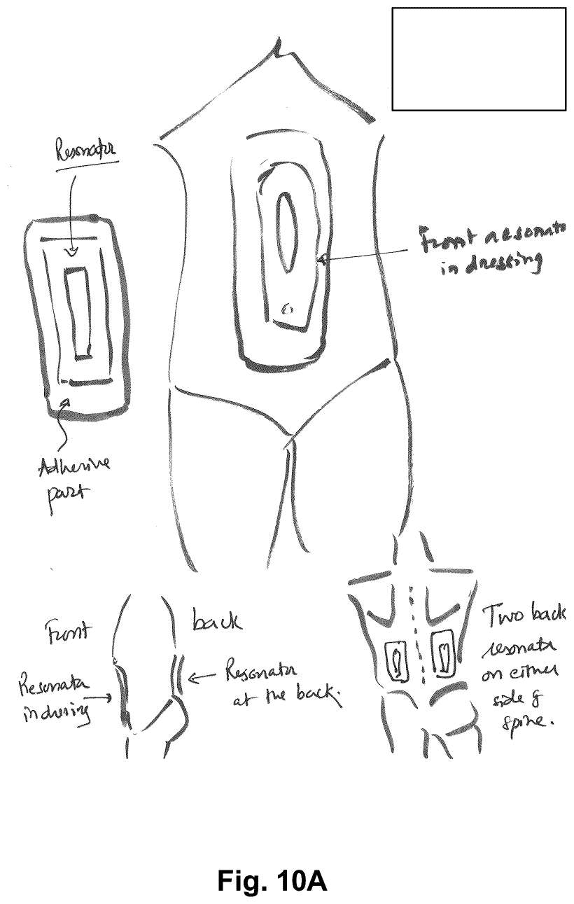

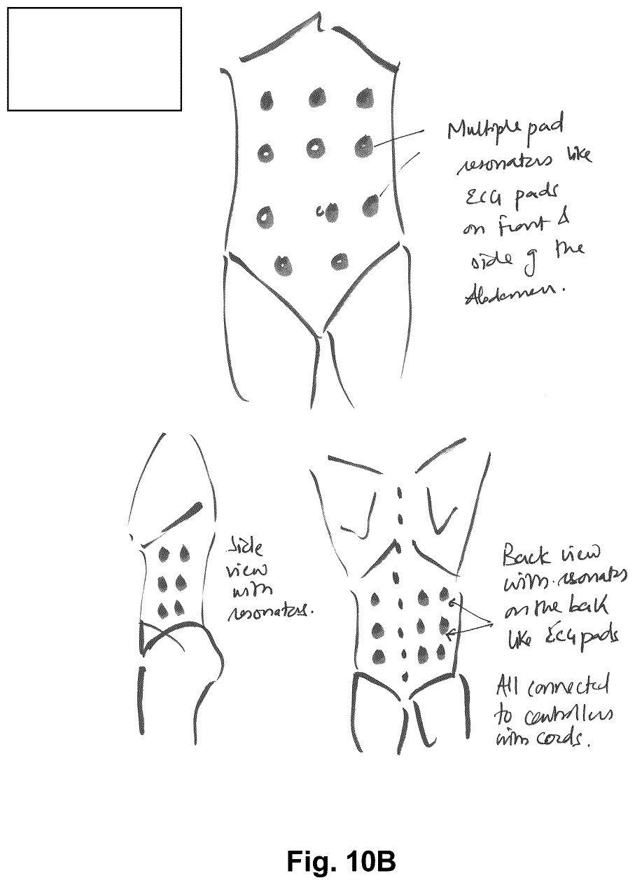

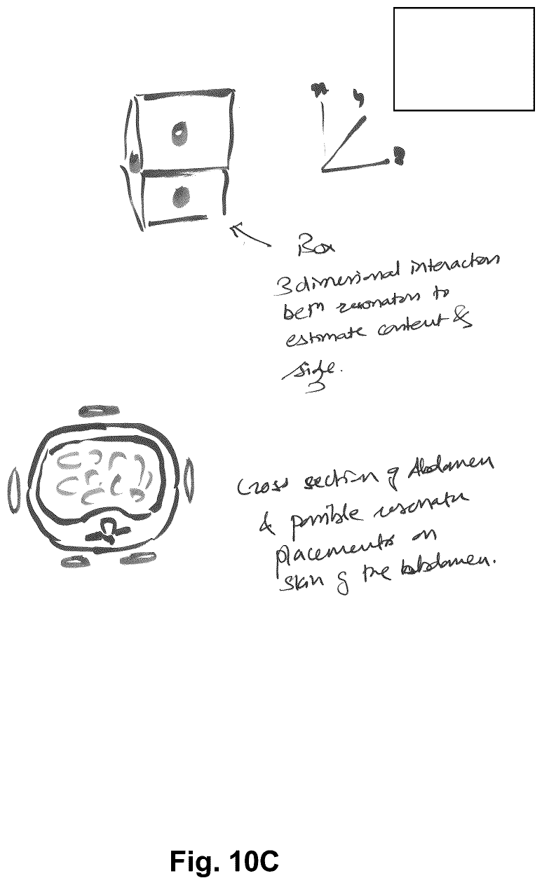

With reference now to FIGS. 10A, 10B and 10C, diagrams of a system utilizing resonators applied to the abdominal wall without a feeding tube inside the patient are shown according to one embodiment. Since the interactions and calculations between two or more resonators gives the same exact information even if they are placed outside the body on skin surface, the principals from above for measuring size and content can be applied to a tubeless system. The skin surface resonators can be for example applied similar to multi-ECG pad-like structures or incorporated in dressings that are already being applied. The resonators can be attached to an adhesive layer. They can alternatively be attached to a flexible elastic garment. In one embodiment, an estimation system includes multiple resonantly coupled resonators configured to attach to a skin surface of the subject, and a controller connected to the plurality of resonantly coupled resonators and configured to estimate a distance between at least a first and second resonantly coupled resonator of the plurality of resonantly coupled resonators based on an electrical signal parameter measured by the first resonantly coupled resonator. The system may included for example a first adhesive patch having one resonator, a pair of resonators, three or more resonators, or for example multiple resonators configured in an array. The system may include for example a second adhesive patch having one resonator, a pair of resonators, three or more resonators, or for example multiple resonators configured in an array. The first and second adhesive patch may for example be configured on opposing sides of the body, adjacent to each other, or on the same side of the body. In one embodiment, the system includes a pair of opposing resonators in front and back and a pair of opposing resonators on either side. A surface may include more resonators than the opposing surface, such as two resonators in the back on either side of the spine, and a single opposing resonator in the front. The resonators can be on separate patches, on a single adhesive layer, or in some other single or combination of apparatus that holds position of the resonator against the body. A body patch or garment-type body suit can be configured with an array of resonators, each connected to the controller. A method for estimation in one embodiment includes the steps of attaching multiple resonantly coupled resonators to a skin surface of the subject and estimating a distance between at least a first and second resonantly coupled resonator of the plurality of resonantly coupled resonators based on an electrical signal parameter measured by the first resonantly coupled resonator.

In one embodiment, estimation of contents is made across at least a vertical and horizontal extent within any hollow or solid viscus including the estimation of medium type, including but not limited to air, water, fluid, blood, CSF, urine, feces, pus or foreign material. Size estimation can be 3-dimensional in all three positive and negative axes (X, Y and Z) for material types. Multi-dimensional estimation can be easily implemented according to the embodiments described above and by selecting pairs of resonators corresponding to the plane(s) or dimension(s) being measured. Accordingly, in one embodiment, an estimation system includes a controller connected to a plurality of resonantly coupled resonators, the controller configured to estimate a multi-dimensional geometry based on an electrical signal parameter measured by plurality of resonantly coupled resonators. In one embodiment, the multi-dimensional content geometry includes a vertical and horizontal component. In one embodiment, the multi-dimensional content geometry includes an X, Y and Z dimension. In one embodiment, the electrical signal parameter is indicative of organ or tissue size, or at least one of air, water, fluid, blood, CSF, urine, feces, pus and foreign material. In one embodiment, an estimation method includes the steps of attaching multiple resonantly coupled resonators to a skin surface of the subject, and estimating a multi-dimensional geometry based on an electrical signal parameter measured by the plurality of resonantly coupled resonators. In one embodiment, the multi-dimensional content geometry includes a vertical and horizontal component. In one embodiment, the multi-dimensional content geometry includes an X, Y and Z dimension. In one embodiment, the electrical signal parameter is indicative of organ or tissue size, or at least one of air, water, fluid, blood, CSF, urine, feces, pus and foreign material.

### REFERENCES

Elke 2015
Elke G, Felbinger T W, Heyland D K. Gastric residual volume in critically ill patients: a dead marker or still alive?. Nutrition in Clinical Practice 2015; 30(1):59-71.
Esteban 2013
controlled study. JPEN. Journal of Parenteral and Enteral Nutrition 2015; 39(4):434-40.
Blaser 2014
Blaser A R, Starkopf J, Kirsimagi U, Deane A M. Definition, prevalence, and outcome of feeding intolerance in intensive care: a systematic review and meta-analysis. Acta Anaesthesiologica Scandinavica 2014; 58(8):914-22.
Booker 2000
Booker K J, Niedringhaus L, Eden B, Arnold J S. Comparison of 2 methods of managing gastric residual volumes from feeding tubes. American Journal of Critical Care 2000; 9(5): 318-24.
Bounoure 2016
Bounoure L, Gomes F, Stanga Z, Keller U, Meier R, Ballmer P, et al. Detection and treatment of medical inpatients with or at-risk of malnutrition: suggested procedures based on validated guidelines. Nutrition 2016; 32(7-8):790-8.
CCPG 2015
Nutrition Critical Care. Canadian Clinical Practice Guidelines. www.criticalcarenutrition.com (accessed 13 Dec. 2017).
Cederholm 2017
Cederholm T, Barazzoni R, Austin P, Ballmer P, Biolo G, Bischoff S C, et al. ESPEN guidelines on definitions and terminology of clinical nutrition. Clinical Nutrition (Edinburgh, Scotland) 2017; 36(1):49-64.
Deane 2007
Deane A, Chapman M J, Fraser R J, Bryant L K, Burgstad C, Nguyen N Q. Mechanisms underlying feed intolerance in the critically ill: implications for treatment. World Journal of Gastroenterology 2007; 13(29):3909-17.
Edwards 2000
Edwards S J, Metheny N A. Measurement of gastric residual volume: state of the science. MEDSURG Nursing 2000; 9 (3):125-8.
Esteban A, Frutos-Vivar F, Muriel A, Ferguson N D, Penuelas O, Abraira V, et al. Evolution of mortality over time in patients receiving mechanical ventilation. American Journal of Respiratory and Critical Care Medicine 2013; 188 (2):220-30.
Feinberg 2017
Feinberg J, Nielsen E E, Korang S K, Halberg Engell K, Nielsen M S, Zhang K, et al. Nutrition support in hospitalised adults at nutritional risk. Cochrane Database of Systematic Reviews 2017, Issue 5. DOI: 10.1002/14651858.CD011598.pub2
Gomes 2017
Gomes F, Schuetz P, Bounoure L, Austin P, Ballesteros-Pomar M, Cederholm T, et al. ESPEN guidelines on nutritional support for polymorbid internal medicine patients. Clinical Nutrition (Edinburgh, Scotland) 2017 Jul. 24 Epub ahead of print]. DOI: 10.1016/j.clnu.^{2017.06.025}
GRADEpro GDT [Computer program]
McMaster University (developed by Evidence Prime). GRADEpro GDT. Hamilton (ON): McMaster University (developed by Evidence Prime), 2015.
Gungabissoon 2015
Gungabissoon U, Hacquoil K, Bains C, Irizarry M, Dukes G, Williamson R, et al. Prevalence, risk factors, clinical consequences, and treatment of enteral feed intolerance during critical illness. JPEN. Journal of Parenteral and Enteral Nutrition 2015; 39(4):441-8.
Hayashi 2014
Hayashi M, Iwasaki T, Yamazaki Y, Takayasu H, Tateno H, Tazawa S, et al. Clinical features and outcomes of aspiration pneumonia compared with non-aspiration pneumonia: a retrospective cohort study. Journal of Infection and Chemotherapy 2014; 20(7):436-42.
Higgins 2003
Higgins J P T, Thompson S G, Deeks J J, Altman D G. Measuring inconsistency in meta-analyses. BMJ 2003; 327 (7414):557-60.
Higgins 2011
Higgins J P T, Green S, editor(s). Cochrane Handbook for Systematic Reviews of Interventions Version 5.1.0 (updated March 2011). The Cochrane Collaboration, 2011. Available from handbook.cochrane.org.
Jeejeebhoy 1977
Jeejeebhoy K N. Definition and mechanisms of diarrhoea. Canadian Medical Association Journal 1977; 116:737-9.
Jeejeebhoy 2002
Jeejeebhoy K N. Short bowel syndrome: a nutritional and medical approach. Canadian Medical Association Journal 2002; 166(10):1297-302.
JSICM 2017
The Committee on Japanese Guidelines for Nutrition Support Therapy in the Adult and Pediatric Critically III Patients, Japanese Society of Intensive Care Medicine. Japanese guidelines for nutrition support therapy in the adult and pediatric critically ill patients. Journal of the Japanese Society of Intensive Care Medicine 2017; 23(2): 185-281. DOI: 10.3918/jsicm. 23.185
Julien 2009
Julien J Y, Martin J G, Ernst P, Olivenstein R, Hamid Q, Lemiere C, et al. Prevalence of obstructive sleep apnea-hypopnea in severe versus moderate asthma. Journal of Allergy and Clinical Immunology 2009; 124(2):371-6.
McClave 2005
McClave S A, Lukan J K, Stefater J A, Lowen C C, Looney S W, Matheson P J, et al. Poor validity of residual volumes as a marker for risk of aspiration in critically ill patients. Critical Care Medicine 2005; 33(2):324-30.
McClave 2009a
McClave S A, Martindale R G, Vanek V W, McCarthy M, Roberts P, Taylor B, et al. Guidelines for the provision and assessment of nutrition support therapy in the adult critically ill patient: Society of Critical Care Medicine (SCCM) and American Society for Parenteral and Enteral Nutrition (ASPEN). JPEN. Journal of Parenteral and Enteral Nutrition 2009; 33(3):277-316.
McClave 2009b
McClave S A, Heyland D K. The physiologic response and associated clinical benefits from provision of early enteral nutrition. Nutrition in Clinical Practice 2009; 24(3):305-15.
McClave 2016a
McClave S A, Taylor B E, Martindale R G, Warren M M, Johnson D R, Braunschweig C, et al. Guidelines for the provision and assessment of nutrition support therapy in the adult critically ill patient: Society of Critical Care Medicine (SCCM) and American Society for Parenteral and Enteral Nutrition (ASPEN). JPEN. Journal of Parenteral and Enteral Nutrition 2016; 40(2):159-211.
McClave 2016b
McClave S A, DiBaise J K, Mullin G E, Martindale R G. ACG Clinical Guideline: Nutrition therapy in the adult hospitalized patient. American Journal of Gastroenterology 2016; 111(3):315-34.
Metheny 2005
Metheny N A, Stewart J, Nuetzel G, Oliver D, Clouse R E. Effect of feeding-tube properties on residual volume measurements in tube-fed patients. JPEN. Journal of Parenteral and Enteral Nutrition 2005; 29(3):192-7.
Metheny 2010
Metheny N A, Davis-Jackson J, Stewart B J. Effectiveness of an aspiration risk-reduction protocol. Nursing Research 2010; 59(1):18-25.
Metheny 2012
Metheny N A, Mills A C, Stewart B J. Monitoring for intolerance to gastric tube feedings: a national survey. American Journal of Critical Care 2012; 21(2):e33-40.
Montejo 1999
Montejo J C. Enteral nutrition-related gastrointestinal complications in critically ill patients: a multicenter study. The Nutritional and Metabolic Working Group of the Spanish Society of Intensive Care Medicine and Coronary Units. Critical Care Medicine 1999; 27(8):1447-53.
Montejo 2010
Montejo J C, Minambres E, Bordeje L, Mesejo A, Acosta J, Heras A, et al. Gastric residual volume during enteral nutrition in ICU patients: the REGANE study. Intensive Care Medicine 2010; 36(8):1386-93.
Moreira 2009
Moreira T V, McQuiggan M. Methods for the assessment of gastric emptying
in critically ill, enterally fed adults. Nutrition in Clinical Practice 2009; 24(2):261-73.
Nguyen 2008
Nguyen N Q, Fraser R J, Bryant L K, Chapman M, Holloway R H. Diminished functional association between proximal and distal gastric motility in critically ill patients. Intensive Care Medicine 2008; 34(7):1246-55.
Nutrition Day
nutritionDay worldwide. nutritionDay worldwide. www.nutritionday.org (accessed 13 Dec. 2017).
Poulard 2010
Poulard F, Dimet J, Martin-Lefevre L, Bontemps F, Fiancette M, Clementi E, et al. Impact of not measuring residual gastric volume in mechanically ventilated patients receiving early enteral feeding: a prospective before-after study.
JPEN. Journal of Parenteral and Enteral Nutrition 2010; 34 (2):125-30.
Powell 1993
Powell K S, Marcuard S P, Farrior E S, Gallagher M L. Aspirating gastric residuals causes occlusion of small-bore feeding tubes. JPEN. Journal of Parenteral and Enteral Nutrition 1993; 17(3):243-6.
Racco 2012
Racco M. An enteral nutrition protocol to improve efficiency in achieving nutritional goals. Critical Care Nurse 2012; 32(4):72-5.
Reignier 2013
Reignier J, Mercier E, Le Gouge A, Boulain T, Desachy A, Bellec F, et al. Effect of not monitoring residual gastric volume on risk of ventilator-associated pneumonia in adults receiving mechanical ventilation and early enteral feeding: a randomized controlled trial. JAMA 2013; 309(3):249-56.
Reintam 2012
Reintam Blaser A, Malbrain M L, Starkopf J, Fruhwald S, Jakob S M, De Waele J, et al. Gastrointestinal function in intensive care patients: terminology, definitions and management. Recommendations of the ESICM Working Group on Abdominal Problems. Intensive Care Medicine 2012; 38(3):384-94.
Reintam 2017
Reintam Blaser A, Starkopf J, Alhazzani W, Berger M M, Casaer M P, Deane A M, et al. Early enteral nutrition in critically ill patients: ESICM clinical practice guidelines. Intensive Care Medicine 2017; 43(3):380-98.
Review Manager 2014 [Computer program]
Copenhagen: Nordic Cochrane Centre: The Cochrane Collaboration. Review Manager 5 (RevMan 5). Version
5.3. Copenhagen: Nordic Cochrane Centre: The Cochrane Collaboration, 2014.
Rubenfeld 2005
Rubenfeld G D, Caldwell E, Peabody E, Weaver J, Martin D P, Neff M, et al. Incidence and outcomes of acute lung injury. New England Journal of Medicine 2005; 353(16): 1685-93.
Shaw 1993
Shaw J H, Koea J B. Metabolic basis for management of the septic surgical patient. World Journal of Surgery 1993; 17(2): 154-64.
Wang 2017
Wang K, Mcllroy K, Plank L D, Petrov M S, Windsor J A. Prevalence, outcomes, and management of enteral tube feeding intolerance: a retrospective cohort study in a tertiary center. JPEN. Journal of Parenteral and Enteral Nutrition 2017; 41(6):959-67.
Wanzer 1989
Wanzer S H, Federman D D, Adelstein S J, Cassel C K, Cassem E H, Cranford R E, et al. The physician's responsibility toward hopelessly ill patients. A second look. New England Journal of Medicine 1989; 320(13):844-9.
Weimann 2017
Weimann A, Braga M, Carli F, Higashiguchi T, Hubner M, Klek S, et al. ESPEN guideline: Clinical nutrition in surgery. Clinical Nutrition (Edinburgh, Scotland) 2017; 36 (3):623-50.
Williams 2010
Williams T A, Leslie G D. Should gastric aspirate be discarded or retained when gastric residual volume is removed from gastric tubes?. Australian Critical Care 2010; 23(4):215-7.
Williams 2014
Williams T A, Leslie G, Mills L, Leen T, Davies H, Hendron D, et al. Frequency of aspirating gastric tubes for patients receiving enteral nutrition in the ICU: a randomized controlled trial. JPEN. Journal of Parenteral and Enteral Nutrition 2014; 38(7):809-16.
Zanetti 2016
Zanetti M. Topic 8 Approach to oral and enteral nutrition (E N) in adults. Module 8.1 Indications, contraindications, complications and monitoring of E N. ESPEN LLL Program. www.testIIInutrition.com/mod III/TOPIC8/m81.pdf (accessed 11 Jan. 2018).
1 Schwarz N T, Beer-Stotz D, Bauer A J. Pathogenesis of para-lytic ileus. Ann. Surg. 2002; 235(1): 31-40.
2 Vather R, O'Grady G, Bissett I P, Dinning P G. Postoperative ileus: mechanisms and future directions for research. Clin. Exp. Pharmacol. Physiol. 2014; 41(05): 358-70.
3 Harnsberger C R, Markel J A, Avavi K. Postoperative ileus. Clin. Colon Rectal Surg. 2019; 32(3): 166-70.
4 Lubawski J, Saclarides T. Postoperative ileus: strategies for reduction. Ther. Clin. Risk Manag. 2008; 4(5): 913-7.
5 Vanek V W, Al-Salti M. Acute pseudo-obstruction of the colon (Ogilvie's syndrome). An analysis of 400 cases. Dis. Colon Rectum 1986; 29(3): 203-10.
6 Stakenborg N, Gomez-Pinilla J, Boeckastaas G E. Postoperative ileus: Pathophysiology. Current therapeutic approaches. Hands Exp. Pharmacol. 2017; 239: 39-57.
7 Schwartz N T, Simmons R L, Bauer A J. Minor intraabdominal injury followed by low dose LPS administration act synergistically to induce ileus. Neurogastroenterol. Motil. 2000; 11(2): 288.
8 Kalff J C, Schrant W H, Simmons R L, Bauer A J. Surgical manipulation of the gut elicits an intestinal muscularis inflammatory response resulting in paralytic ileus. Ann. Surg. 1998; 228: 625-53.
9 Deitch E A, Specian R D, Berg R D. Endotoxin-induced bacterial translocation and mucosal permeability: role of xanthineoxidase, complement activation, and macrophage products. Crit. Care Med. 1991; 19: 785-91.
10 Frank P G, Lisanti M P. ICAM-1: role in inflammation and in the regulation of vascular permeability. Am. J. Physiol. Heart.
11 Bragg D, E I-Sharkawy A M, Psaltis E, Maxwell-Armstrong C_{A}, Lobo D N. Postoperative ileus: recent developments inpathophysiology and management. Clin. Nutr. 2015; 34(03):367-76.
12 Grass F, Slieker J, Jurt J, et al. Postoperative ileus in an enhanced recovery pathway—a retrospective cohort study. Int. J. Colorectal. Dis. 2017; 32(05): 675-81.
13 Hughes M J, Ventham N T, McNally S, Harrison E, WigmoreS. Analgesia after open abdominal surgery in the setting of enhanced recovery surgery: a systematic review and meta-analysis. JAMA Surg. 2014; 149(12): 1224-30.
14 Halabi W J, Kang C Y, Nguyen V Q, et al. Epidural analgesiain laparoscopic colorectal surgery: a nationwide analysis of use and outcomes. JAMA Surg. 2014; 149(02): 130-6.
15 daSilva M, Lomelin D, Tsui J, Klinginsmith M, Tadaki C, Langenfeld S. Pain control for laparoscopic colectomy: analysis is of the incidence and utility of epidural analgesia compared to conventional analgesia. Tech. Coloprocto1.2015; 19(09): 515-20.
16 Liu H, Hu X, Duan X, Wu J. Thoracic epidural analgesia (TEA) vs. patient controlled analgesia (PCA) in laparoscopic colectomy: a meta-analysis. Hepatogastroenterology 2014; 61(133): 1213-9.
17 Shum N F, Choi H K, Mak J C, Foo D C, Li W C, Law W L. Randomized clinical trial of chewing gum after laparoscopic colorectal resection. Br. J. Surg. 2016; 103(11): 1447-52.
18 Schwenk W, B€ohm B, Haase O, Junghans T, M€uller J M. Laparoscopic versus conventional colorectal resection: a prospective randomised study of postoperative ileus and earlypostoperative feeding. Langenbecks Arch. Surg. 1998; 383(01): 49-55.
19 Fujii S, Ishibe A, Ota M, et al. Short-term results of a randomized study between laparoscopic and open surgery in elderly colorectal cancer patients. Surg. Endosc. 2014; 28(02): 466-76.
20 Ng W Q, Neill J. Evidence for early oral feeding of patients after elective open colorectal surgery: a literature review. J. Clin. Nurs. 2006; 15(06): 696-709.
21 Carmichael J C, Keller D S, Baldini G, et al. Clinical practice guidelines for enhanced recovery after colon and rectal surgery from the American Society of Colon and Rectal Surgeons and Society of American Gastrointestinal and Endoscopic Surgeons. Dis. Colon Rectum 2017; 60(08): 761-84.
22 Wolthuis A M, Bislenghi G, Fieuws S, de Buck van Overstraeten A, Boeckxstaens G, D'Hoore A. Incidence of prolonged postoperative ileus after colorectal surgery: a sys-tematic review and meta-analysis. Colorectal Dis. 2016; 18(01): O1-O9.
23 Northover J M A. Intestinal surgery. In: Postoperative complications in surgery. Eds Alan V Pollock, Mary Evans, Black-well scientific publications 1991 London.
24 Artinyan A, Nunoo-Mensah J W, Balasubramaniam S, et al. Prolonged postoperative ileus-definition, risk factors, and predictors after surgery. World J. Surg. 2008; 32(07):1495-500.
25 Vather R, Josephson R, Jaung R, Robertson J, Bissett I. Development of a risk stratification system for the occurrence of prolonged postoperative ileus after colorectal surgery: a prospective risk factor analysis. Surgery 2015; 157(04): 764-73.
26 Vather R, Josephson R, Jaung R, Kahokehr A, Sammour T, Bissett I. Gastrografin in prolonged postoperative ileus: a double-blinded randomized controlled trial. Ann. Surg. 2015; 262(01): 23-30.
27 Todd S R, Marshall G T, Tyroch A H. Acute gastric dilatation revisited. The American Surgeon 2000; 65(8): 709-10.
28 Magee C, Blackwell V, Travis S. Toxic dilatation of the colon. Medicine 2015; 43(3): 171-3.
29 Frager D H, Baer J W, Rothpearl A, Bossart P A. “Distinction between postoperative ileus and mechanical small-bowel obstruction: value of C T compared with clinical and other radiographic findings”. AJR Am. J. Roentgenol. 1995; 164(4): 891-4.
30 Jangjoo A, Mohammadipoor F, Fazel A, Mehrabi Bahar M, Aliakbarian M, Jabbari Nooghabi M. The role of nasogastric intubation on postoperative gastrointestinal function in patients with obstructive jaundice. Indian J. Surg. 2012; 74(5): 376-80.
31 Koukouras D, Mastronikolis N S, Tzoracoleftherakis E, Ange-lopoulou E, Kalfarentzos F, Androulakis J. The role of naso-gastric tube after elective abdominal surgery. Clin. Ter. 2001; 152: 241-4.
32 Cutillo G, Maneschi F, Franchi M, Giannice R, Scambia G, Benedetti-Panici P. Early feeding compared with nasogastric decompression after major oncologic gynecologic surgery: a randomized study. Obstet. Gynecol. 1999; 93: 41-5. https://doi.org/10.1016/S0029-7844(98)00401-3.].
33 Wittbrodt E. The impact of postoperative ileus and emerging therapies. Pharm. Treatment 2006; 31: 39-59. ©2020 The Authors. Acute Medicine & Surgery published by John Wiley & Sons Australia, Ltd on behalf of Japanese Association for Acute Medicine Acute Medicine & Surgery2020; 7:e573Paralytic ileus 5 of5

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. An internal estimation system comprising:
a flexible tube having a first resonator, the flexible tube configured to advance into an anatomical lumen of a subject;
a second resonator configured to attach to a skin surface of the subject, wherein the first and second resonator are resonantly coupled; and
a controller connected to the first resonator and the second resonator, wherein the controller is configured to estimate a distance between the first and second resonator based on an electrical signal parameter measured by the first resonator.

2. The system of claim 1, wherein the electrical signal parameter is signal amplitude.

3. The system of claim 1, wherein the electrical signal parameter is signal frequency.

4. The system of claim 1, wherein the electrical signal parameter is signal quality.

5. The system of claim 1, wherein the controller is configured to estimate a parameter of content within the anatomical lumen based on the electrical signal parameter.

6. The system of claim 5, wherein the content is one of air, liquid and solid contents.

7. The system of claim 1, wherein the first resonator is one of a plurality of resonators attached to the flexible tube.

8. The system of claim 1, wherein the second resonator is one of a plurality of resonators attached to the skin surface of the subject.

9. The system of claim 1, wherein the first resonator has a spiral geometry.

10. The system of claim 1, wherein the first resonator is attached to an outer wall of the flexible tube.

12. The system of claim 1, wherein the second resonator is planar.

13. The system of claim 1, wherein the second resonator has a radially disposed perimeter comprising a plurality of alternating concave and convex curves.

15. The system of claim 1, wherein at least one of the first and second resonator comprises a copper wire winding.

16. The system of claim 1, wherein at least one of the first and second resonator comprises a polyamide surface layer.

17. A method for internal estimation comprising:
advancing a flexible tube having a first resonator into an anatomical lumen of a subject;
attaching a second resonator to a skin surface of the subject, wherein the first and second resonator are resonantly coupled; and
estimating a distance between the first and second resonator based on an electrical signal parameter measured by the first resonator.

18. The method of claim 17, wherein the electrical signal parameter is signal amplitude.

19. The method of claim 17, wherein the electrical signal parameter is signal frequency.

20. The method of claim 17, wherein the electrical signal parameter is signal quality.

21. The method of claim 17 further comprising:
estimating a parameter of content within the anatomical lumen based on the electrical signal parameter.

22. The method of claim 21, wherein the content is one of air, liquid and solid contents.

23. The method of claim 17, wherein the first resonator is one of a plurality of resonators attached to the flexible tube.

24. The method of claim 17, wherein the second resonator is one of a plurality of resonators attached to the skin surface of the subject.

25. The method of claim 17, wherein the first resonator has a spiral geometry.

26. The method of claim 17, wherein the first resonator is attached to an outer wall of the flexible tube.

27. The method of claim 17, wherein the second resonator is planar.

28. The method of claim 17, wherein the second resonator has a radially disposed perimeter comprising a plurality of alternating concave and convex curves.

29. The method of claim 17, wherein at least one of the first and second resonator comprise a copper wire winding.

30. The method of claim 17, wherein at least one of the first and second resonator comprise a polyamide surface layer.

31. An estimation system comprising:
a plurality of resonantly coupled resonators configured to attach to a skin surface of the subject; and
a controller connected to the plurality of resonantly coupled resonators and configured to estimate a distance between at least a first and second resonantly coupled resonator of the plurality of resonantly coupled resonators based on an electrical signal parameter measured by the first resonantly coupled resonator.

32. The system of claim 31, wherein the electrical signal parameter is signal amplitude.

33. The system of claim 31, wherein the electrical signal parameter is signal frequency.

34. The system of claim 31, wherein the electrical signal parameter is signal quality.

35. The system of claim 31, wherein the controller is configured to estimate a parameter of content within the anatomical lumen based on the electrical signal parameter.

36. The system of claim 35, wherein the content is one of air, liquid and solid contents.

37. The system of claim 31, wherein the second resonator is planar.

38. The system of claim 31, wherein the second resonator has a radially disposed perimeter comprising a plurality of alternating concave and convex curves.

39. A method for estimation comprising:
attaching a plurality of resonantly coupled resonators to a skin surface of the subject; and
estimating a distance between at least a first and second resonantly coupled resonator of the plurality of resonantly coupled resonators based on an electrical signal parameter measured by the first resonantly coupled resonator.

40. The method of claim 39, wherein the electrical signal parameter is signal amplitude.

41. The method of claim 39, wherein the electrical signal parameter is signal frequency.

42. The method of claim 39, wherein the electrical signal parameter is signal quality.

43. The method of claim 39 further comprising:
estimating a parameter of content within the anatomical lumen based on the electrical signal parameter.

44. The method of claim 39, wherein the content is one of air, liquid and solid contents.

45. The method of claim 39, wherein the second resonator is planar.

46. The method of claim 39, wherein the second resonator has a radially disposed perimeter comprising a plurality of alternating concave and convex curves.

47. An estimation system comprising:
plurality of resonantly coupled resonators; and
a controller connected to the plurality of resonantly coupled resonators and configured to estimate a multi-dimensional geometry based on a plurality of electrical signal parameters measured by the plurality of resonantly coupled resonators.

48. The system of claim 47, wherein the multi-dimensional geometry includes a vertical and horizontal component.

49. The system of claim 47, wherein the multi-dimensional geometry includes an X, Y and Z dimension.

50. The system of claim 47, wherein the electrical signal parameter is indicative of an organ or tissue size.

51. The system of claim 47, wherein the electrical signal parameter is indicative of at least one of air, water, fluid, blood, CSF, urine, feces, pus and foreign material.

52. A method of estimation method comprising:
positioning a plurality of resonantly coupled resonators near an anatomical structure of a subject; and
estimating a multi-dimensional geometry based on a plurality of electrical signal parameters measured by the plurality of resonantly coupled resonators.

53. The method of claim 52, wherein the multi-dimensional geometry includes a vertical and horizontal component.

54. The method of claim 52, wherein the multi-dimensional geometry includes an X, Y and Z dimension.

55. The method of claim 52, wherein the electrical signal parameter is indicative of an organ or tissue size.

56. The method of claim 52, wherein the electrical signal parameter is indicative of at least one of air, water, fluid, blood, CSF, urine, feces, pus and foreign material.
